(19) United States
(12) Patent Application Publication
(10) Pub. No.: US 2010/0137267 A1
(43) Pub. Date: Jun. 3, 2010

(54) FORMULATIONS FOR TREATMENT OF ADIPOSE TISSUE, CUTANEOUS TISSUE AND DISORDERS, AND MUSCULAR TISSUE

(76) Inventor: John Daniel Dobak, La Jolla, CA (US)

Correspondence Address: WILSON, SONSINI, GOODRICH & ROSATI, 650 PAGE MILL ROAD, PALO ALTO, CA, 94304-1050, US

(21) Appl. No.: 12/445,571

(22) PCT Filed: Oct. 16, 2007
(86) PCT No.: PCT/US07/81568

§ 371 Date: Jan. 22, 2010

Related U.S. Application Data
(60) Provisional application No. 60/898,009, filed on Jan. 29, 2007.
(60) Provisional application No. 60/852,221, filed on Oct. 17, 2006.
(60) Provisional application No. 60/919,011, filed on Mar. 20, 2007.

(51) Int. Cl. A61K 31/567; A61P 3/04

(52) U.S. Cl. 514/171

(57) ABSTRACT

Compositions, formulations, methods, and systems for treating regional fat deposits and fat-related conditions, dermal conditions, and muscular conditions. Methods comprise administering a composition comprising at least one compound that reduces desensitization of beta adrenergic receptors, for example a glucocorticosteroid, and/or at least one long-acting beta-2 adrenergic receptor agonist, for example, formoterol. Compositions to be administered include sustained release formulations comprising at least one long-acting beta-2 adrenergic receptor agonist, at least one compound that reduces desensitization of beta adrenergic receptors, or both in a sustained crystalline microparticle form.

### CROSS-REFERENCE

This application claims the benefit of U.S. Provisional Application Nos. 60/852,221 filed Oct. 17, 2006; 60/898,009 filed Jan. 29, 2007; and 60/919,011, filed Mar. 20, 2007, which applications are incorporated herein by reference in their entirety.

### BACKGROUND OF THE INVENTION

Excess body fat is a severe health care issue in modern societies. Chronic health conditions promoted by excess body fat include, e.g., cardiovascular disease and diabetes mellitus type 2. Moreover, excess body fat greatly undermines personal appearance and self image.

Accumulation of fat stores can occur unevenly in the body. For example, some persons may accumulate fat predominantly in the abdominal cavity while others predominately in the subcutaneous tissue. Gender differences may also be apparent with women accumulating fat in the thighs and lateral buttocks and males in the waist. Women may accumulate fatty deposits of the thighs, which have a rumpled or “peau-de-orange” appearance, resulting in a condition referred to as cellulite. Cellulite may be related to skin architecture which allows subdermal fat herniation, sometimes referred to as adipose papillae. Other factors that may be related to cellulite include altered and/or reduced connective tissue septae, vascular and lymph changes that lead to fluid accumulation, and inflammation. Fat tissue may also accumulate in the form of a fibrous fatty deposit known as a lipoma. Utilization of fat stores may occur unevenly. Persons who have lost substantial weight may still have regional pockets of fat accumulation that are resistant to reduction unless unhealthy extremes of weight loss are achieved. Exercise may affect subcutaneous fat stores differently, with deeper tissues responding with lipolysis and superficial stores being more resistant. Cellulite may also still be present despite weight loss, and lipomas are typically not affected by weight loss.

### SUMMARY OF THE INVENTION

Adipose tissue is the primary energy storage tissue of the body. Fat cells, or adipocytes, store this energy in the form of triglycerides. Triglycerides are mobilized from fat stores to provide caloric energy to the body through hormonal induction of triglyceride hydrolysis. This process releases free or non-esterified fatty acids and glycerol into the blood for use by other body tissues. The breakdown of triglycerides from fat store is referred to as lipolysis. Growth of new adipocytes also occurs, which is referred to as adipogenesis. Primary hormones and neurotransmitters that control lipolysis in the body are the catecholamines. Adipose tissue has beta-1, 2, and 3 adrenergic receptors and alpha-2 adrenergic receptors. Binding of beta adrenergic receptor agonists (“beta adrenergic agonists”) to beta adrenergic (“beta”) receptors in adipose tissue results in adipocyte lipolysis, while binding of alpha receptor agonists inhibits lipolysis. Beta adrenergic receptor activation also inhibits adipogenesis. In humans, the beta-2 receptor are often the most abundant on fat cell surfaces and the primary mediator of beta adrenergic receptor-stimulated lipolysis. Stimulation of lipolysis by beta adrenergic agonists is mediated by adenylate cyclase and increased formation of cyclic adenosine monophosphate (cyclic AMP, cAMP).

Longer term exposure of adipocytes to beta adrenergic agonists results in receptor desensitization and down regulation, and a loss of lipolytic activity. Accordingly, described herein are compositions, formulations, methods, and systems for treating adipocyte deposits by contacting a targeted fat deposit with a composition comprising at least one long-acting beta-2 adrenergic receptor agonist and a compound that reduces desensitization of the target tissue to the long-acting beta-2 adrenergic receptor agonist, e.g., at least one glucocorticosteroid, antihistamine, or any combination thereof.

In one aspect provided herein is a method for reducing a regional fat deposit in a subject in need thereof (e.g., a subject suffering from obesity) comprising administering to the subject, a sustained release pharmaceutical composition comprising a therapeutically effective amount of at least one compound for reducing desensitization of beta adrenergic receptors. In some embodiments, the sustained release pharmaceutical composition is administered by a parenteral, topical, intramuscular, transdermal, transvascular, subcutaneous, or orbital route of administration. In some embodiments, the at least one compound comprises a glucocorticosteroid, an antihistamine, or a combination thereof. In some embodiments, the at least one compound comprises dexamethasone, prednisolone, methylprednisolone, fluticasone propionate, budesonide, ketotifen, or any combination thereof. In some embodiments, the therapeutically effective amount of the at least one compound comprises a crystalline microparticle suspension of the at least one compound. In some embodiments, the therapeutically effective amount of the at least one compound is released for about 12 hours to about 45 days (e.g., about 3 days to about 10 days). In some embodiments, the above-mentioned sustained release pharmaceutical composition further comprises a therapeutically effective amount of at least one beta adrenergic agonist that is selective for the beta-2 adrenergic receptor and is formulated as a crystalline microparticle suspension. In some embodiments, the onset of release of the at least one beta adrenergic agonist is delayed relative to the onset of release of the at least one compound. In some embodiments, a liposuction procedure is prescribed to or performed on the subject treated with the just-mentioned sustained release pharmaceutical composition. In some embodiments, the sustained release pharmaceutical composition further comprises a therapeutically effective amount of the at least one beta adrenergic agonist in solubilized form.

In some embodiments, the method further comprises administering, in addition to the at least one compound for reducing desensitization of beta adrenergic receptors, a composition comprising a therapeutically effective amount of at least one beta adrenergic agonist that is selective for the beta-2 adrenergic receptor (e.g., salmeterol, formoterol, bambuterol, eformoterol, isoproterenol, albuterol, fenoterol, or any combination thereof). In some embodiments, the therapeutically effective amount of the at least one beta adrenergic agonist is administered to the subject about one day to about two weeks after administering the at least one compound. In some embodiments, the at least one compound for reducing desensitization of beta adrenergic receptors is administered by injection prior to administration of the just-described composition comprising a therapeutically effective amount of at least one beta adrenergic agonist. In some embodiments, the composition comprising a therapeutically effective amount of the at least one beta adrenergic agonist is administered orally. In some embodiments, the composition comprising the therapeutically effective amount of the at least one beta adrenergic agonist is administered as a crystalline microparticle suspension.

In another aspect provided herein is a method for performing liposuction, comprising performing liposuction on a subject in need thereof that has been administered a sustained release pharmaceutical composition comprising a therapeutically effective amount of at least one compound for reducing desensitization of beta adrenergic receptors and a therapeutically effective amount of at least one beta adrenergic agonist that is selective for the beta-2 adrenergic receptor.

In a further aspect provided herein is a method for reducing a regional fat deposit in a subject in need thereof comprising administering to the subject a therapeutically effective amount of one or more adrenergic receptor pathway-stimulating compounds (e.g., a catecholamine, an alpha adrenergic antagonist, forskolin, aminophylline, analogs thereof, or any combination thereof); and a therapeutically effective amount of at least one compound for reducing beta adrenergic receptor desensitization (e.g., in the form of a crystalline microparticle suspension). In some embodiments, the just-described method further comprises administering a composition comprising a therapeutically effective amount of at least one beta adrenergic agonist that is selective for the beta-2 adrenergic receptor. In some embodiments, the above-described composition further comprises a therapeutically effective amount of at least one compound for reducing beta adrenergic receptor desensitization. In some embodiments, the composition comprising the therapeutically effective amount of the at least one beta adrenergic agonist is administered about one day to two weeks after the at least one compound. In some embodiments, the therapeutically effective amount of the one or more adrenergic receptor pathway-stimulating compounds and the therapeutically effective amount of the at least one beta adrenergic agonist that is selective for the beta-2 adrenergic receptor are coadministered.

In yet another aspect provided herein is a sustained release pharmaceutical composition (e.g., an injectable formulation) comprising a therapeutically effective amount of at least one compound for reducing desensitization of beta adrenergic receptors, wherein the therapeutically effective amount of the at least one compound comprises a crystalline microparticle suspension of the at least one compound. In some embodiments, the at least one compound is a glucocorticosteroid, an antihistamine, or any combination thereof. In some embodiments, the at least one compound comprises dexamethasone, prednisolone, methylprednisolone, fluticasone propionate, budesonide, ketotifen, or any combination thereof. In some embodiments, the sustained release pharmaceutical composition further comprises a crystalline microparticle suspension of at least one beta adrenergic agonist that is selective for the beta-2 adrenergic receptor (e.g., salmeterol, formoterol, bambuterol, eformoterol, isoproterenol, albuterol, or fenoterol, or any combination thereof). In some embodiments, the sustained release pharmaceutical composition comprising a crystalline microparticle suspension of the at least one beta adrenergic agonist, further comprises a therapeutically effective amount of the at least one beta adrenergic agonist in solubilized form. In some embodiments, the release rate of the at least one beta adrenergic agonist is slower than the release rate of the at least one compound for reducing desensitization of beta adrenergic receptors. In some embodiments, the above-described sustained release pharmaceutical composition, in addition to comprising at least one compound for reducing beta adrenergic receptor desensitization and at least one beta adrenergic agonist that is selective for the beta-2 adrenergic receptor, further comprises a therapeutically effective amount of a thyroid hormone (e.g., T3 or T4).

In another aspect provided herein is a method for increasing muscle mass in a subject in need thereof, comprising administering to the subject a sustained release pharmaceutical composition comprising a therapeutically effective amount of at least one compound for reducing desensitization of beta adrenergic receptors and a therapeutically effective amount of at least one beta adrenergic agonist.

In a further aspect provided herein is a method for treating a dermal condition in a subject in need thereof, comprising administering to the subject a sustained release pharmaceutical composition comprising a therapeutically effective amount of at least one compound for reducing desensitization of beta adrenergic receptors and a therapeutically effective amount of at least one beta adrenergic agonist.

In still another aspect provided herein is a method for treating obstructive sleep apnea in a subject in need thereof, comprising administering to the subject a sustained release pharmaceutical composition comprising a therapeutically effective amount of at least one compound for reducing desensitization of beta adrenergic receptors and a therapeutically effective amount of at least one beta adrenergic agonist.

In yet another aspect provided herein is a method for treating strabismus in a subject in need thereof, comprising administering to the subject a sustained release pharmaceutical composition comprising a therapeutically effective amount of at least one compound for reducing desensitization of beta adrenergic receptors and a therapeutically effective amount of at least one beta adrenergic agonist.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic illustration of adipocyte lipolysis

FIG. 2 is a bar graph illustrating the dose-dependent induction of lipolysis in cultured adipocytes by the long-acting beta-2 agonist Formoterol after a three hour incubation.

FIG. 3 is a bar graph illustrating the dose-dependent induction of lipolysis in cultured adipocytes by the long-acting beta-2 agonist Salmeterol after a three hour incubation.

FIG. 4 is a bar graph illustrating the dose-dependent induction of lipolysis in cultured adipocytes by the glucocorticosteroid Budesonide after a short incubation period (three hours), and the suppression of lipolysis after longer incubation periods (18 hours).

FIG. 5 is a bar graph illustrating the dose-dependent suppression of lipolysis in cultured adipocytes by the long-acting beta-2 agonist Salmeterol given alone for 18 hours, and the dose-dependent induction of lipolysis by Salmeterol after 18 hours when given in combination with the glucocorticosteroid Budesonide.

FIG. 6 is a bar graph illustrating average within-animal differences in epididymal fat pad mass (left fat pad versus right fat pad) in fat pads injected with vehicle solution (2% PEG), Formoterol alone, or Formoterol plus Budesonide over three day treatment period.

FIG. 7 is a bar graph illustrating the dose-dependent reduction of fat pad mass for two different dose combinations of the beta-2 agonist Formoterol and the glucocorticosteroid Budesonide over a three day treatment period (administration on alternate days).

FIG. 8 is a bar graph illustrating the dose-dependent reduction of epididymal and inguinal fat pad masses by the beta-2 agonist Formoterol in combination with a fixed dose of the glucocorticosteroid Budesonide over a three day treatment period.

FIG. 9 is a bar graph illustrating the dose-dependent reduction of epididymal fat pad mass by the beta-2 agonist Formoterol in combination with a fixed dose of the glucocorticosteroid Methylprednisolone over a three day treatment period.

FIG. 10 is a bar graph showing the reduction in fat pad mass, reduction in average fat cell diameter, and the reduction in fat cell number after administration of the formulation. The reduction in cell number is primarily seen after 2 weeks of treatment, and is consistent with the need for additional treatment time for these affects to become measurable. These results are consistent with the mechanism of action of the formulation. Q3D=Every third day injection for 2 weeks. QOD=Every other day injection for 1 week.

FIG. 11 is a bar graph showing the fat loss expected with human equivalent dosing (projected values based on rodent data). Daily human equivalent dosing, e.g., through a sustained release formulation, is expected to produce more than two times the fat mass reduction of the QOD dose. Q3D=Every third day injection for 2 weeks. QOD=Injection every other day for 1 week.

### DETAILED DESCRIPTION OF THE PREFERRED EMBODIMENT

Catecholamines are the primary regulators of adipose tissue through the adrenergic receptors. Adipose tissue has beta-1, 2, and 3 adrenergic receptors and alpha-2 adrenergic receptors. Binding of beta adrenergic agonists to beta adrenergic receptors in adipose tissue can result in adipocyte lipolysis, while binding of alpha receptor agonists can inhibit lipolysis. Beta adrenergic receptor activation can also inhibit adipogenesis. In humans, the beta-2 receptor are often the most abundant on fat cell surfaces and the primary mediator of beta adrenergic receptor-stimulated lipolysis. Stimulation of lipolysis by beta adrenergic agonists is mediated by adenylate cyclase and increased formation of cyclic adenosine monophosphate (cyclic AMP, cAMP). Alpha 2 receptors reduce lipolysis in mature fat cells. Alpha-2 adrenergic receptors may be involved in the proliferation of pre-adipocytes. Glucocorticosteroids increase responses (e.g., lipolysis) to catecholamine stimulation. This action is likely due to up-regulation of beta-adrenergic receptors and other components involved in secondary intracellular messengers.

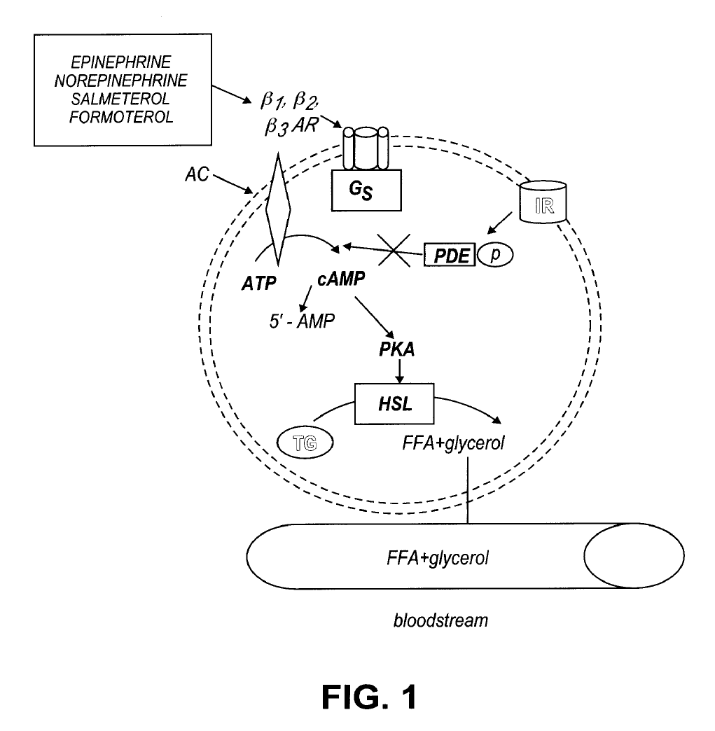

Provided herein are pharmaceutical compositions, formulations, methods, and systems to achieve regional fat, adipose tissue, and adipocyte reduction therapy through adrenergic system modulation. As used herein, the term “modulation” is generally used in its usual sense, and more particularly to refer to adrenergic receptor agonism, adrenergic receptor antagonism, and/or changes in receptor signaling pathways. One example of a change in receptor signaling pathways includes an increase in cyclic AMP, for example as illustrated schematically in FIG. 1. In some embodiments, modulation refers to receptor upregulation or an increase in the number of adrenergic receptors, a decrease in receptor deactivation or sequestration, receptor activity changes (for example, an increase in activity), and/or changes in receptor affinity.

While not wishing to be bound by theory, it is believed that in some embodiments of the methods described herein, sustained modulation of adrenergic receptors in adipose tissue results in a combination of sustained lipolysis, reduced lipid content of adipocytes, reduced adipocyte cell size, reduced adipose tissue mass or fat accumulation, and/or improved cosmetic appearance. Again, without wishing to be bound by theory, it is also believed that sustained adrenergic modulation results in sustained inhibition of fat cell proliferation (adipogenesis). Accordingly, in some embodiments, the methods described herein provide selective reduction of regional and/or subcutaneous accumulations of adipose tissue and adipocytes, including cellulite, through sustained adrenergic modulation. In some embodiments, the compositions described herein are useful for treating cellulitic fat accumulation and/or lipomas. In some embodiments, the subject to be treated is diagnosed as obese, e.g., having more than 25% body (men) or more than 30% body fat (women).

In various embodiments, one or more long-acting selective beta-2 adrenergic receptor agonists is administered separately or in combination with one or more compounds that reduce desensitization of the target tissue to the beta-adrenergic receptor agonist(s), for example, glucocorticosteroids or ketotifen, or analogs thereof. The term desensitization includes both short term desensitization (tachyphylaxis), as well as long term desensitization, as well as desensitization over other time periods. Beta-2 adrenergic receptor agonists are also referred to herein as “beta-2 agonists” and “beta-2 receptor agonists.” Unless otherwise specified, references to beta-2 adrenergic receptor agonists also include their analogs, physiologically acceptable salts and/or solvates. Some embodiments of the composition comprise from about 100:1 to about 1:100 long-acting selective beta-2 agonist to glucocorticosteroid.

As discussed above, lipolytic activity and adipocyte proliferation inhibition are believed to be mediated through modulation of adrenergic receptors in adipose tissue and/or on adipocytes. In some embodiments, the reduction therapy is enhanced through prolonged exposure or sustained activity of one or more adrenergic receptor agonists and/or receptor pathway stimulating compounds, for example, catecholamines, beta adrenergic agonists, alpha antagonists, forskolin, aminophylline, analogs thereof, or combinations thereof.

Some embodiments provide sustained adrenergic modulation through the use of pharmaceutical compositions comprising one or more long-acting substantially selective beta-2 receptor agonists. Some embodiments of the sustained activity pharmaceutical composition comprise one or more suitable long-acting, selective beta-2 agonists, for example, salmeterol 1, formoterol 2, bambuterol 3, physiologically acceptable salts or solvates thereof, or combinations thereof.

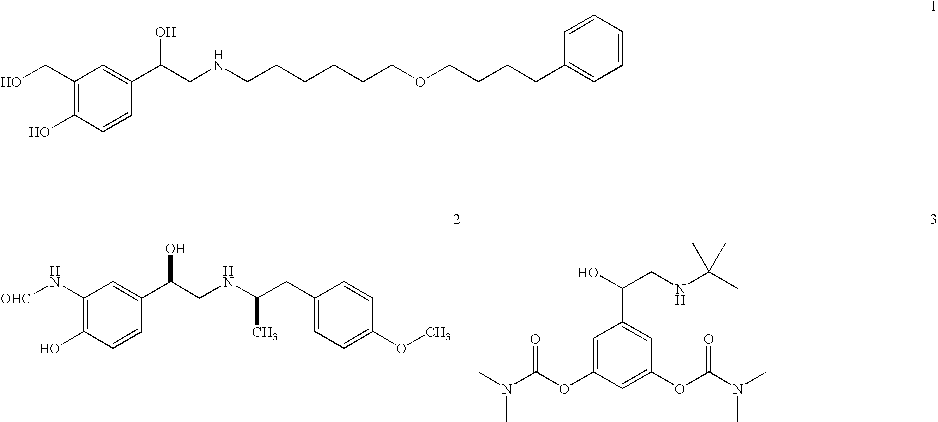

Sustained adrenergic modulation is not observed with typical adrenergic compositions because the adrenergic compound is generally rapidly removed from the adipose tissue through the blood and/or lymph in part due to their hydrophilicity. Furthermore, long term exposure of adipose tissue to adrenergic agents, particularly beta adrenergic receptor agonists, results in receptor desensitization through receptor phosphorylation and sequestration. These effects limit the ability of an adrenergic modulating composition to treat adipose tissue and result in tachyphylaxis, a condition in which the body experiences a rapidly decreasing response to the agonist following administration of the initial doses, to the desired lipolytic and anti adipogenesis effect. Consequently, the treatment effect is short lived.

Short-acting beta-2 agonists often result in tachyphylaxis, as discussed above. However, because preferred embodiments of long-acting selective beta-2 agonists have substantially selective beta-2 receptor activity and high lipophilicity, the activities of long-acting beta-2 agonists continue for longer periods of time in adipose tissue compared with short-acting beta-2 agonists. Partial beta-2 receptor antagonist activity, which occurs with the use of salmeterol, prevents some desensitization that occurs with continuous exposure of adipocytes to full adrenergic agonists. Further, salmeterol may not completely activate the arrestin signaling that leads to receptor internalization and degradation and leads to long term receptor down regulation. Compared with short-acting beta-2 agonists, lipolysis also occurs for a longer time after administration because long-acting selective beta-2 agonists have longer half-lives. The combination of longer half-lives and activities reduces the required frequency of administration of the pharmaceutical compositions. Consequently, in some embodiments, daily administration or more than once daily administration of the composition is not required. Moreover, preferred embodiments of long-acting selective beta-2 agonists also exhibit greater selectivity for beta-2 receptors, permitting substantially similar therapeutic effects compound with short-acting beta-2 agonists at a lower dosage. Further the more selective beta-2 activity can limit cardiac side effects, which are often induced by beta-1 receptor stimulation in the heart.

As discussed above, lipolysis and/or inhibition of adipogenesis and lipid accumulation are stimulated by the beta-1, 2, or 3 receptor subtypes. Thus, agonists to one, two and/or all three receptors are capable of stimulating lipolysis and/or inhibition of adipogenesis. In humans, beta-2 receptor activity is believed to be more important for stimulating lipolysis, particularly in the presence of an anti-inflammatory steroid or glucocorticosteroid.

Long-acting selective beta-2 agonists, for example, salmeterol 1 (±2-(hydroxymethyl)-4-[1-hydroxy-2-[6-(4-phenylbutoxy)hexylamino]ethyl]-phenol, CAS Reg. No. 94749-08-3), and formoterol 2 (±N-[2-hydroxy-5-[1-hydroxy-2-[1-(4-methoxyphenyl)propan-2-ylamino]ethyl]phenyl]methanamide, CAS Reg. No. 73573-87-2), are preferred in some embodiments. Some embodiments of the compositions comprise one or more long-acting selective beta-2 agonists as physiologically acceptable salts or solvates, for example, salmeterol xinafoate and/or formoterol fumarate. In many cases, salts and/or solvates of a beta-2 agonists will have the desired activity. Unless otherwise specified, references to an active ingredient, for example, to salmeterol 1, formoterol 2, isoproterenol 4, albuterol 5, fenoterol, and forskolin, include the compounds themselves as well as a physiologically acceptable analogs, salts, and/or solvates thereof, or combinations thereof.

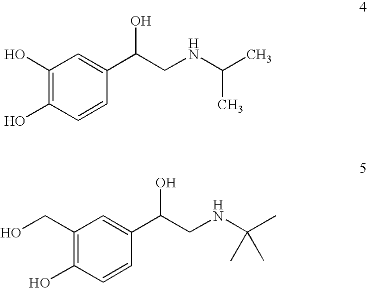

Some preferred long-acting beta adrenergic agonists exhibit high intrinsic adenylate cyclase activity, which increase cAMP synthesis. For example, some embodiments comprise formoterol 2 as a long-acting beta-2 selective agonist, which exhibits some combination of higher potency, reduced systemic effects, high intrinsic activation of adenylate cyclase, and/or increases in cyclic AMP, a mediator of lipolysis.

In some preferred embodiments formoterol 2 is present as a physiologically acceptable salt and/or solvate thereof. Suitable physiologically acceptable salts of formoterol 2 include, for example, acid addition salts derived from inorganic and organic acids, such as the hydrochloride, hydrobromide, sulfate, phosphate, maleate, fumarate, tartrate, citrate, benzoate, 4-methoxybenzoate, 2-hydroxybenzoate, 4-hydroxybenzoate, 4-chlorobenzoate, p-toluenesulphonate, methanesulphonate, ascorbate, salicylate, acetate succinate, lactate, glutarate, gluconate, tricarballylate, hydroxynaphthalenecarboxylate, oleate, combinations thereof, and the like. Preferred embodiments comprise formoterol 2 as its fumarate salt and/or as a dihydrate. Suitable tissue concentration of formoterol 2 for adipose tissue treatment include from about 1 pM to about 100 μM, e.g., about 0.01 μM to about 50 μM, 0.5 μM to about 50 μM, about 2.0 μM to about 50 μM μM, about 5 μM to about 50 μM, about 10 μM to about 50 μM, about 20 μM to about 75 μM, or any other tissue concentration of formoterol from about 0.1 nM to about 100 μM.

In some embodiments, salmeterol is used in the compositions and methods described herein. Salmeterol 1 exhibits partial agonist activity, which is believed to reduce receptor desensitization and may limit arrestin signaling leading to less receptor down regulation. In some embodiments salmeterol 1 is present as a physiologically acceptable salt and/or solvate thereof. Suitable physiologically acceptable salts of salmeterol 1 include, but are not limited to acid addition salts derived from inorganic and organic acids, such as the hydrochloride, hydrobromide, sulfate, phosphate, maleate, tartrate, citrate, benzoate, 4-methoxybenzoate, 2-hydroxybenzoate, 4-hydroxybenzoate, 4-chlorobenzoate, p-toluenesulphonate, methanesulphonate, ascorbate, salicylate, acetate, fumarate, succinate, lactate, glutarate, gluconate, tricarballylate, hydroxynaphthalenecarboxylate, 1-hydroxy-2-naphthalenecarboxylate, 3-hydroxy-2-naphthalenecarboxylate, oleate, combinations thereof, and the like. In some embodiments salmeterol 1 is provided as the 1-hydroxy-2-naphthalene carboxylate salt (hydroxynaphthoate).

In some embodiments, a suitable tissue concentration of salmeterol 1 for adipose tissue treatment ranges from about 1 pM to about 100 μM, preferably from about 1.0 nM to about 1 μM, e.g., about 10 nM to about 1 μM, about 40 nM to about 3 μM, about 0.1 μM to about 1 μM, or any other tissue concentration of salmeterol from about 1.0 nM to about 10 uM.

In some embodiments, a long-acting selective beta-2 agonist to be administered is formoterol and a therapeutically effective amount of formoterol is about 0.001 μg/day to about 100 μg/day, e.g., about 0.001 μg/day to about 50 μg/day, 0.01 μg/day to about 1.0 μg/day, about 0.1 μg/day to about 10 μg/day, about 1 μg/day to about 20 μg/day, about 5 μg/day to about 40 μg/day, about 25 μg/day to about 75 μg/day, about 50 μg/day to about 100 μg/day of formoterol, or any other dose of formoterol from about 0.001 μg/day to about 100 μg/day.

In some embodiments, a long-acting selective beta-2 agonist to be administered is salmeterol and a therapeutically effective amount of salmeterol to be administered is about 0.01 μg/day to about 1000 μg/day, e.g., about 0.1 μg/day to about 100 μg/day, about 1 μg/day to about 100 μg/day, about 10 μg/day to about 100 μg/day, about 50 μg/day to about 100 μg/day, or any other dose of salmeterol from about 0.01 μg/day to about 1000 μg/day.

A “therapeutically effective amount,” as used herein, refer to a sufficient amount of an agent (e.g., a long-acting beta 2 agonist) or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case can be determined using techniques, such as a dose escalation study. The term “therapeutically effective amount” includes, for example, a prophylactically effective amount. An “effective amount” of a compound disclosed herein, such as a selective beta-2 agonist used alone or in combination with other compounds (e.g., a compound for reducing beta-2 adrenergic receptor desensitization), is an amount effective to achieve a desired pharmacologic effect or therapeutic improvement without undue adverse side effects. It is to be understood that “an effect amount” or “a therapeutically effective amount” can vary from subject to subject, due to variation in metabolism of beta-2 agonists and compounds used in combination with beta-2 agonists (e.g., glucocorticosteroids), age, weight, general condition of the subject, the condition being treated, the severity of the condition being treated, and the judgment of the prescribing physician.

As used herein, the term “coadministered,” refers to the administration of two or more therapeutic agents in a single formulation or separate formulations or routes of administration in any order for the purpose of treating the same health condition (e.g., a lipoma) in the same subject.

Some embodiments comprise optically pure isomers of the beta adrenergic agonist(s), which improve lipolysis and adipogenesis inhibition and reduce potential side effects. In some embodiments, these optically pure isomers allow formulations comprising larger loadings of an active ingredient, for example, by eliminating one or more isomers with no physiological effect, a lesser a physiological effect, a negative effect, and/or an undermined physiological effect. Removing the undesired bounds of a racemic mixture isolates the active isomer, or eutomer, thereby allowing more eutomer to be loaded in a give formulation by removing the inactive components.

Two stereogenic centers in a molecule generally generate two diastereomers, referred to herein as (R*,R*) and (R*,S*), and their enantiomers. Diastereomers are stereoisomers that are not enantiomers, that is, the mirror image of one diastereomer is not superimposable on another diastereomer. Enantiomers are stereoisomers that are mirror images of each other. A racemate is a 1:1 mixture of enantiomers. The enantiomers of the (R*,R*) diastereomers are referred to as the (R,R) and (S,S) enantiomers, which are mirror images of each other and therefore share some chemical and physical properties, for example melting points. Similarly, the (R,S) and (S,R) isomers are enantiomers of the (R*,S*) enantiomer. For example, formoterol 2 is available as a racemate of the (R,R)- and (S,S)-isomers in a 1:1 ratio, typically as the dihydrate of the fumarate salt. Some embodiments comprise the (R,R) enantiomer, (R,R)-formoterol, which is more active as a long-acting beta-2 agonist. Some embodiments comprise optically pure isomers of other beta-2 agonists, for example, (R)-salmeterol.

Additionally, in some embodiments, at least one long-acting selective beta-2 agonists is highly lipophilic, thereby providing a pharmaceutical composition with sustained activity in fat tissue. It is believed that high lipid solubility extends the residence time of the beta-2 agonist in the adipose tissue, thereby eliminating or reducing the need for a sustained release and/or controlled release carrier in some embodiments. In formulations comprising a sustained release carrier, for example, a sustained release polymer, the high lipophilicity of the beta-2 agonist facilitates incorporation into the sustained release carrier, as discussed in greater detail below.

Salmeterol 1 and formoterol 2 have high lipid solubilities, which extends their residence time in the adipose tissue and/or in one or more adipose cells. Some embodiments of the composition comprise a highly lipophilic beta adrenergic agonist, which reduces or eliminates the need for a sustained or controlled release carrier due to partitioning and sequestration in the adipose tissue thereby prolonging the treatment effect. In some embodiments, beta adrenergic agonists with an oil-water partition coefficient of at least about 1000 or at least about 10,000 to 1 are used. For example, salmeterol 1 is at least 10,000 times more lipophilic than albuterol 5, a short acting hydrophilic beta adrenergic agonist.

Sustained beta adrenergic activity is further enhanced by preventing or reducing desensitization (tachyphylaxis) that can occur with continuous exposure of adipocytes to adrenergic agonists as discussed above. “Compounds that reduce desensitization of beta-adrenergic receptors” (e.g., reduce desensitization of a target tissue to a beta adrenergic agonist) include all suitable compounds that reduce tolerance of the target tissue to the beta-adrenergic receptor agonists, including glucocorticosteroids and suitable antihistamines, for example, ketotifen, and thyroid hormones, for example T3 and T4. Glucocorticosteroids are also referred herein as “anti-inflammatory steroids,” “glucocorticosteroids,” and/or “corticosteroids.” Glucocorticosteroids are believed to sensitize regional fat accumulations by increasing the number of surface beta-2 receptors, thereby favoring lipolysis or fat reduction over fat storage. Glucocorticosteroids also decrease the number of alpha-2 receptors. Glucocorticosteroids also stabilize or reduce receptor down regulation especially when given simultaneously with a beta adrenergic agonist. Of note, estrogen can induce the expression of alpha-2 adrenergic receptors in subcutaneous adipose tissue in women resulting in a ratio of beta-2 receptor to alpha-2 receptor of less than 1. A ratio of beta-2 receptors to alpha-2 receptors greater than about 1 is believed to cause fat reduction rather than fat accumulation in adipocytes.

Some embodiments of the composition comprising one or more glucocorticosteroids are effective in treating regions of fat comprising a reduced number of beta-2 receptors and or an increased number of alpha-2 receptors, which are resistant to beta adrenergic stimulation of lipolysis or inhibition of adipogenesis, for example, subcutaneous adipose tissue, especially in women.

Without wishing to be bound by theory, it is believed that glucocorticosteroids or other compounds for reducing desensitization of beta adrenergic receptors increase lipolysis, adipogenesis inhibition, and/or regional fat reduction during beta adrenergic agonist exposure. Thus, in some embodiments, a therapeutically effective amount of a compound (e.g., a glucocorticosteroid) for reducing desensitization of beta-adrenergic receptors is administered to increase lipolytic activity and/or increase the number of beta-receptors in the target tissue, and thereby increase fat deposit reduction. In some embodiments, a patient is administered a sustained release pharmaceutical composition comprising a therapeutically effective amount of at least one compound for reducing desensitization of beta adrenergic receptors. For example, the compound for reducing desensitization of beta adrenergic receptors can be formulated as a crystalline microparticle suspension so as to provide extended release over a desired time period from about 12 hours to about 12 months, e.g., one day, 3 days, 7 days, 10 days, 1 month, 45 days, 2 months, 3 months, 4 months, 6 months, 7 months, 8 months, 9 months, 10 months, 11 months, or any other time period from about 12 hours to about 12 months. In some embodiments, the sustained release pharmaceutical composition further comprises a therapeutically effective amount of at least one beta adrenergic agonist that is selective for the beta-2 adrenergic receptor (e.g., salmeterol or formoterol). In some embodiments, the at least one beta adrenergic agonist is in the formulated as a crystalline microparticle suspension for extended release. In some embodiments, the crystalline microparticles have a diameter ranging from about 5 μm to about 150 μm, e.g., 10 μm to 130 μm, 20 μm to 100 μm, 30 μm to 75 μm, or any other diameter ranging from about 5 μm to about 150 μm. In some embodiments, the average particle diameter is about 10 μm to about 50 μm, e.g., about 15 μm, 20 μm, 25 μm, 30 μm, 40 μm, or any other average diameter from about 10 μm to about 50 μm. In some embodiments, the beta adrenergic agonist is formulated so as to have a release profile that is delayed relative to the release of the compound for reducing desensitization of beta adrenergic receptors.

In some embodiments, a compound for reducing desensitization of beta adrenergic receptors is a glucocorticosteroid. Examples of suitable corticosteroids include dexamethasone 6, fluticasone propionate 7, budesonide 8, prednisolone 9, methylprednisolone 10, and their analogs. In some embodiments, the glucocorticosteroid is dexamethasone. In some embodiments, the corticosteroid is methylprednisolone. 6(9-fluoro-11,17-dihydroxy-17-(2-hydroxyacetyl)-10,13,16-trimethyl-6,7,8,11,12,14,15,16-octahydrocycl openta[a]phenanthren-3-one, CAS Reg. No. 50-02-2) and/or fluticasone propionate 7.

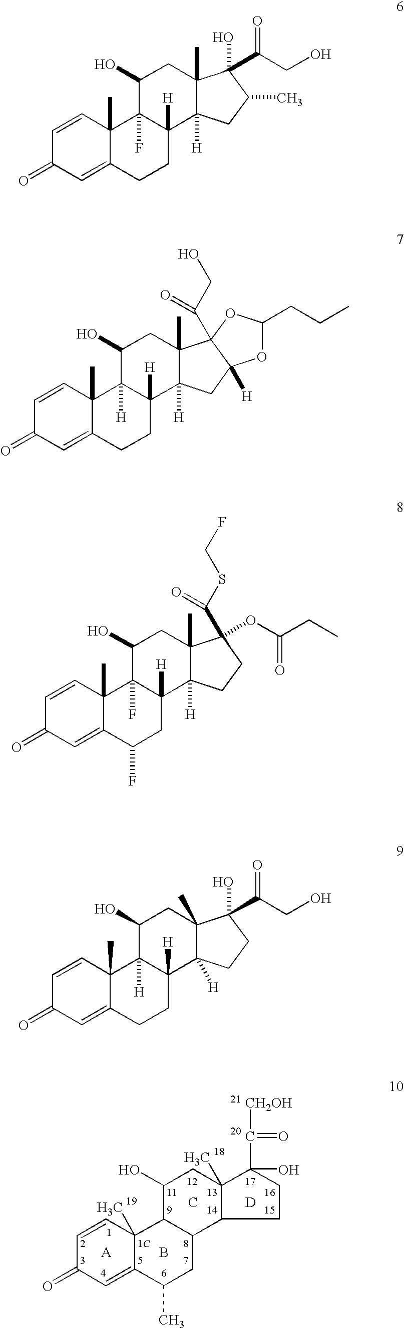

As discussed above, in some embodiments a suitable compound for reducing beta adrenergic receptor desensitization is ketotifen 11, which is also useful as an antihistamine. Some embodiments of the composition comprise one compound that reduces desensitization of the adipose tissue to the beta-2 agonist.

In some embodiments a plurality of compounds for reducing beta adrenergic receptor desensitization are used, for example, a plurality of glucocorticosteroids. Some preferred embodiments comprise at least one glucocorticosteroid and the antihistamine ketotifen or an analog of ketotifen.

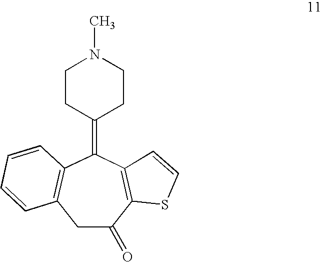

In some embodiments, beta-2 receptor activity or density increases in adipocytes within a regional fat deposit in response to corticosteroid or ketotifen administration, particularly in the presence of a beta adrenergic agonist. In some embodiments, increasing beta-2 receptor activity and/or density potentiates the effect of long- and short-acting beta-2 agonists. Thus, in some embodiments, the glucocorticosteroid sensitizes adipose tissue in a regional fat deposit to the effects of beta-2 receptor stimulation, e.g., lipolysis, inhibition of adipogenesis, and/or apoptosis, and/or increases the ratio of beta-2 adrenergic receptors to alpha-2 adrenergic receptors, thereby shifting the balance of the adipose tissue from fat accumulation to fat loss and resulting in reduction of the regional fat deposit. In some embodiments beta-2 receptor number is increased or maintained especially with a glucocorticosteroid or ketotifen.

Appropriate tissue concentrations of glucocorticosteroids used for the therapeutic methods described herein range from about 0.001 μM to about 10 mM, e.g., from about 1.0 μM to about 5 mM, from about 40 μM to about 3 mM, from about 100 μM to about 1 mM, or any other tissue concentration of the glucocorticosteroid from about 10 μM to about 10 mM.

In some embodiments, a glucocorticosteroid to be administered is budesonide and the pharmaceutically effective amount of budesonide is about 1.0 to about 320 μg/day, e.g., about 80 μg/day to about 300 μg/day, about 100 μg/day to about 280 μg/day, about 120 μg/day to about 260 μg/day, about 140 μg/day to about 240 μg/day, about 160 μg/day to about 220 μg/day, about 180 μg/day to about 200 μg/day, about 185 μg/day to about 195 μg/day of budesonide, or any other dose of budesonide from about 60 μg/day to about 320 μg/day.

In some embodiments, the glucocorticosteroid to be administered is fluticasone and the therapeutically effective amount of fluticasone is from about 1.0 μg/day to about 500 μg/day, e.g., about 120 μg/day to about 480 μg/day, about 140 μg/day to about 460 μg/day, about 160 μg/day to about 440 μg/day, about 180 μg/day to about 420 μg/day, about 200 μg/day to about 400 μg/day, about 220 μg/day to about 380 μg/day, about 240 μg/day to about 360 μg/day, about 260 μg/day to about 340 μg/day, about 275 μg/day to about 310 μg/day, or about 290 μg/day to about 300 μg/day of fluticasone, or any other dose of fluticasone from about 100 μg/day to about 500 μg/day.

In some embodiments, the glucocorticosteroid to be administered is methylprednisolone at about 1.0 μg/day to 10,000 μg/day or more, e.g., 50 μg/day to 5,000 μg/day, 100 μg/day to 5,000 μg/day, 500 μg/day to 5000 μg/day, 700 μg/day to 3,000 μg/day, 800 μg/day to 2500 μg/day, 1000 μg/day to 2000 μg/day, or any other dose from about 1.0 μg/day to 10,000 μg/day. In some embodiments methylprednisolone succinate is solubilized in or coadministered with crystalline microparticle methylprednisolone acetate suspension to provide immediate dosing and sustained dosing.

In some embodiments, a thyroid hormone is used to increase the number of beta-2 adrenergic receptors.

Some embodiments of the composition comprise one or more anti-lipolytic blocking agents (i.e., agents that disinhibit lipolysis), for example, selective alpha-2 receptor antagonists such as phentolamine 12 (CAS Reg. No. 73-05-2) or yohimbine 13 (CAS Reg. No. 146-48-5) block anti-lipolytic effects in regional fat accumulation. Anti-lipolytic effects in adipocytes and adipose tissue are typically observed in subcutaneous and regional areas of fat accumulation. For example, when exposed to beta adrenergic agonists, subcutaneous fat has a lower lipolytic rate than visceral fat. In some embodiments, exposing orbital fat to anti-lipolytic blocking agents improves lipolytic activity in some embodiments.

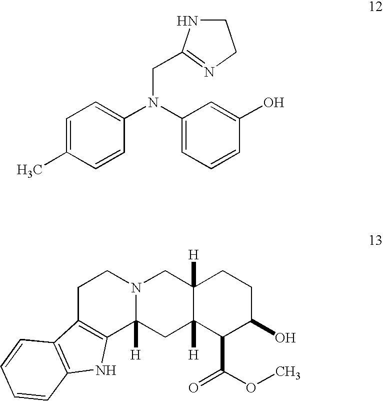

Some embodiments of the composition comprise adrenergic receptor pathway-stimulating compounds that enhance the effect of a long-acting selective beta-2 agonist. For example, aminophylline 14 (1,3-dimethyl-7H-purine-2,6-dione, diethylamine CAS Reg. No. 317-34-0) and theophylline 15 (CAS Reg. No. 58-55-9) are lipolytic agent that block the breakdown of cyclic AMP.

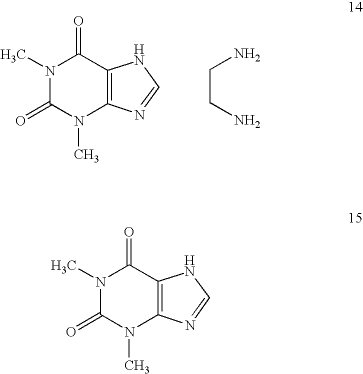

Other optional ingredients increase the secondary signals created by the beta adrenergic agonist binding. For example, in some embodiments, the composition comprises forskolin 16 (CAS Reg. No. 66575-29-9), which stimulates adenylate cyclase, thereby increasing the synthesis of cyclic AMP initiated by the long-acting beta adrenergic agonist. The increased concentration of cyclic AMP helps sustain lipolytic activity.

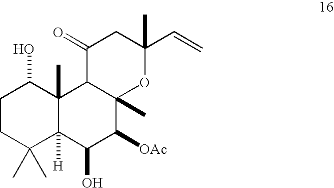

In some embodiments the compositions described herein further comprises one or more nonselective beta adrenergic agonists, for example, isoproterenol 4, and/or short-acting selective beta-2 agonists, for example, terbutaline. Some compositions comprise at least one of an alpha-2 antagonist, or physiologically acceptable salts or solvates thereof. Some embodiments of the composition comprise growth hormone in combination with a long-acting beta adrenergic agonist and glucocorticosteroid, which appears to stimulate lipolysis.

In some embodiments, in addition to treating a subject with any of the compositions described herein, a physician or other authorized medical caregiver prescribes a liposuction procedure to a subject, or performs a liposuction procedure on the subject to further reduce regional fat deposits.

In some embodiments, a liposuction procedure is performed on a subject to whom has been administered a sustained release pharmaceutical composition comprising a therapeutically effective amount of at least one compound for reducing desensitization of beta adrenergic receptors (e.g., a glucocorticosteroid) or a sustained release pharmaceutical composition comprising both a beta adrenergic agonist that is selective for beta-2 receptors and a therapeutically effective amount of at least one compound for reducing desensitization of beta adrenergic receptors. Without wishing to be bound by theory, administering any of the just-described sustained release pharmaceutical compositions to a subject prior to liposuction is likely to increase the efficacy of the liposuction procedure.

Embodiments of the composition are formulated for administered by any suitable method, for example, as described in Remington: The Science And Practice Of Pharmacy (21st ed., Lippincott Williams & Wilkins). Exemplary routes of administration include, but are not limited to parenteral, oral, subcutaneous, topical, intramuscular, transdermal, transmucosal, sublingual, intranasal, transvascular, subcutaneous, orbital, or respiratory. In some embodiments, the composition is formulated for injection of an area at which treatment is desired, for example, in a regional fat deposit.

In some embodiments, beta adrenergic agonists, compounds for preventing beta adrenergic receptor desensitization, or both are formulated as crystalline microparticle suspensions to prolong release and thereby further sustain adrenergic modulation.

Any suitable pharmaceutically acceptable excipient appropriate for a particular route of administration can be used. Examples of pharmaceutically acceptable carriers include, but are not limited to, buffers, saline, or other aqueous media. The compounds of the invention are preferably soluble in the carrier which is employed for their administration (e.g., subcutaneous). Alternatively, a suspension of the active compound or compounds (e.g., a suspension of crystalline microparticles) in a suitable carrier is employed. In some embodiments, one or more of the beta-2 receptor agonists or glucocorticosteroids are formulated in a liquid carrier, for example, as a solution, a suspension, a gel, and/or an emulsion. Some embodiments comprise any suitable lipophilic carrier, for example, modified oils (e.g., Cremophor® BASF, Germany), soybean oil, propylene glycol, polyethylene glycol, derivatized polyethers, combinations thereof, and the like. Some embodiments comprise a microparticulate and/or nanoparticulate carrier for at least one of the beta-2 receptor agonists and/or glucocorticosteroids, as discussed in greater detail below. Some embodiments comprise one or more sustained or controlled release carriers or agents, for example, polymer microspheres. Some embodiments comprise excipients suitable for stable suspensions for micronized particles of the beta-2 receptor agonists or glucocorticosteroids.

Injectable formulations are administered using any method known in the art, for example, using a single needle, multiple needles, and/or using a needleless injection device. In some embodiments, a tissue loading dose of the active ingredients formulated in a suitable carrier delivered by injection. In some embodiments, delivery comprises single needle injection. In some embodiments, delivery comprises injection using a multi-needle array, which, in some embodiments, provides a wide dispersion of the formulation in the target tissue. In some embodiments, formulations are injected in a manner that allows dispersal into the appropriate layer of subcutaneous fat in areas where regional fat.

In some embodiments, the beta-2 agonist and the compound that reduces desensitization are administered, for example injected, as separate formulations, or, alternatively, are administered by separate routes administered orally followed by injection of a long-acting beta-2 agonist. In some embodiments, the compound that reduces desensitization is administered prior to the beta-2 agonist. In other embodiments, the beta-2 agonist is administered prior to the compound that reduces desensitization.

The interval between administration of the compound that reduces desensitization and administration of the beta-2 agonist can be an interval from about 5 minutes to about 1 month, e.g., 30 minutes, 1 hour, 6 hours, 12 hours, one day, 2 day, 3 days, 4 days, 5 days, 6 days, 7 days, 10 days, 2 weeks, 3 weeks, or any other time interval from about 5 minutes to about 1 month. In a preferred embodiment, the compound that reduces desensitization (e.g., a corticosteroid) is administered orally up to about 7 days, e.g., 3 days, 4 days, 5 days, 6 days, 7 days, 8 days, 9 days, or 10 days prior to administering the beta-2 agonist (e.g., by local injection of a crystalline microparticle suspension formulation into a fat deposit).

In other embodiments, the beta-2 agonist is co-administered (e.g., as part of the same formulation) with the compound that reduces beta adrenergic receptor desensitization (e.g., a glucocorticosteroid).

In some embodiments a formulation, a subject to be treated is provided a depot formulation, which comprises one or more sustained or controlled release agents for providing a sustained or controlled release of a beta-2 agonist or the compound (e.g., a glucocorticosteroid) for inhibiting desensitization of beta adrenergic receptors. In such formulations, the beta-2 agonist, the compound for reducing beta adrenergic receptor desensitization, or both are encapsulated in, bound to, and/or conjugated to the sustained or controlled release agent or carrier. In some embodiments, biocompatible, biodegradable sustained or controlled release formulations provide local tissue activity. Sustained release can be over a period from about 12 hours to about 12 months, e.g., one day, 3 days, 7 days, 10 days, 1 month, 45 days, 2 months, 3 months, 4 months, 6 months, 8 months, 9 months, 10 months, 11 months, or any other time period from about 12 hours to about 12 months. Suitable sustained or controlled release agents or carriers include polymers, macromolecules, active ingredient conjugates, hydrogels, contaminations thereof, and the like. Some embodiments of the sustained release carrier target fat, for example, liposomes. Preferably, the sustained release materials are selected to facilitate delivery of a substantially equal amount of the active substance per unit time. Several rounds of injections of the sustained release formulation can be made over time to treat a single area. In some embodiments, sustained release results from formulating the beta-2 agonist, a compound for reducing beta adrenergic receptor desensitization, or both as a suspension of crystalline drug microparticles.

In some embodiments, the sustained release agent comprises a polymer, for example, polylactides, polyglycolides, poly(lactide glycolides) polylactic acids, polyglycolic acids, polyanhydrides, polyorthoesters, polyetheresters, polycaprolactones, polyesteramides, polycarbonates, polycyanoacrylates, polyurethanes, polyacrylates, and blends, mixtures, or copolymers of the above, which are used to encapsulate, binds, or conjugate with the active ingredients(s) (e.g., beta adrenergic agonists and/or glucocorticosteroids). Some preferred embodiments of sustained release polymers comprise polyethylene glycol groups to which one or more of the active ingredients is conjugated. In some preferred embodiments, the sustained release agent comprises poly(lactide glycolide) (PLGA, poly(lactic-co-glycolic acid)) copolymer 17.

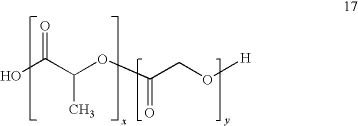

Some embodiments of the sustained release agent comprise one or more hydrogels, including modified alginates. Examples of suitable modified alginates include those disclosed in WO 98/12228. Some embodiments of the sustained release agent comprise an albumin-based nano-particle carrier or excipient.

In some embodiments, a formulation comprising a prepolymer solution is injected into the target tissue site, where it is then polymerized (e.g., by photopolymerization) or solidified (e.g., by using temperature sensitive gelling materials) in vivo.

In some embodiments, the controlled release materials have release characteristics designed for the particular application of tissue reduction. In some embodiments, the sustained release or controlled release agent is formed into microparticles, such as microspheres, which are formulated as an injectable solution and/or gel. In some embodiments, the microparticles range in size from about 10 μm to about 100 μm in diameter (e.g., about 15 μm, 20 μm, 25 μm, 30 μm, 40 μm, 50 μm, 60 μm, 70 μm, 80 μm, 90 μm or any other diameter from about 10 μm to about 100 μm). In some embodiments, the microparticles are uniform in size. In other embodiments, the microparticles vary in size by about 10% to about 300%, e.g., 30%, 40%, 50%, 70%, 80%, 90%, 120%, 150%, 170%, 190%, 200%, 225%, 250%, 275%, or by any other percentage variation in size from about 10% to about 300%. In some embodiments, formulations comprising alginates and/or poly(lactide-co-glycolide)s 17 are provided as an injectable gel or processed into microspheres. In other embodiments, the beta-2 agonist or a corticosteroid (or other compound for reducing beta adrenergic receptor desensitization) are formed as crystalline microparticles. Other examples of suitable injectable biodegradable, biocompatible materials suitable for microparticle formation include chitosan, dextran, hydroxyapatite, and silicon.

Microspheres and/or microparticles are formed using any method, including by solvent evaporation and/or emulsion polymerization. In some embodiments, the microspheres have average diameters of from about 5 μm to about 60 μm, preferably, about 20 μm. In some embodiments, PLGA is manufactured with varying ratios of lactide to glycolide depending on the desired rate of release of the active ingredient(s). Because the rate of degradation of this copolymer is proportional to its crystallinity and the proportion of glycolide in the formulation, non-racemic mixtures of the lactide and/or glycolide increase crystallinity and slow the rate of degradation. Higher proportions of glycolide increase the rate of degradation. In some embodiments, a ratio of about 65%-75% lactide to about 25%-35% glycolide provides active ingredients released over from about 2 weeks to about 45 days. In other embodiments, the ratio of lactide to glycolide is from about 0:100 to about 100:0, thereby providing other release rates.

Some embodiments of the microspheres or microparticles comprise hollow and/or porous interiors. In some embodiments, the microspheres comprise a solid or porous outer shell.

In some embodiments, formulations comprising a porous outer shell and/or microsphere exhibit a biphasic release profile of the active ingredient(s) with an initial release burst of the active ingredient(s), followed by a sustained release associated with degradation of the polymeric microspheres. While not wishing to be bound by theory, it is thought that the initial release burst loads the tissue with an effective lipolytic/adipogenesis inhibitory concentration of the active ingredient(s), with the subsequent slower release maintaining the desired concentration. In some embodiments, the different microsphere structures and active ingredient release profiles optimize the treatment effect of fat deposit tissue and adipocytes and through adrenergic receptor modulation. For example, in formulations comprising both a beta adrenergic agonist and a compound for reducing beta adrenergic receptor desensitization, the beta adrenergic agonist can be formulated so the onset of its release is delayed, i.e., slower, relative to that of the compound for reducing desensitization, e.g., by about one day to about two weeks, e.g., 2 days, 3 days, 4 days, 5 days, 6 days, 7 days, 10 days, 12 days, or any other delay period from about one day to about two weeks. I compound In some preferred embodiments, sustained local tissue concentrations of long-acting selective beta-2 adrenergic agents, such as salmeterol 1 and/or formoterol 2 are at about 1.0 pM to about 10 μM, e.g., about 0.01 μM to about 10 μM, about 0.1 μM to about 5 μM, 0.5 μM to about 4 μM, or any other concentration from about 0.001 μM to about 10 μM. In various embodiments, suitable sustained local tissue concentrations of glucocorticosteroids can range from about 0.01 μM to 10 mM.

In some embodiments, one or more of the active ingredients are encapsulated, bound, and/or conjugated to the polymer at a ratio of about 10-12% by mass compared to the polymer microspheres. The amount of active ingredient as a mass percentage of the carrier (e.g., microparticles or microspheres) is referred to herein as “active ingredient loading.” As used herein, the terms “loaded” and “loading” refer to active ingredients substantially encapsulated bound, and/or conjugated to a carrier. In some embodiments, the active ingredient loading is up to about 75%. Thus, some preferred formulations comprise one or more beta-2 adrenergically active ingredients, such as salmeterol 1, formoterol 2, and/or their physiologically acceptable salts and solvates, loaded on polymer microspheres at about 1 mg to about 20 mg of active ingredient (e.g., about 2 mg, 5 mg, 7 mg, 10 mg, 12 mg, 14 mg, 15 mg, 17 mg, 18 mg, or any other amount of active ingredient from about 1 mg to about 20 mg) per about 10 mg to about 200 milligrams of polymer. In some embodiments, a formulation with this active ingredient loading is sufficient for providing from about 12 hours to about 45 days (e.g., about 3 days, 7 days, 16 days, 20 days, 25 days, 30 days, 35 days, 40 days, 42 days, or any other period from about 12 hours to about 45 days) of active ingredient release at a concentration suitable to produce lipolysis and/or adipogenesis inhibition as described herein. Similarly, in some embodiments, the glucocorticosteroids budesonide and fluticasone in pharmaceutically acceptable forms are loaded with about 1 mg to about 20 mg of active ingredient per about 10 to about 200 mg of polymer.

In some embodiments, two or more active ingredients are loaded into the same microparticle, for example, in a liposome or PLGA. Thus, some embodiments, a polymer encapsulating a glucocorticosteroid in the adrenergic compound is delivered simultaneously to the adipose tissue. Alternatively, the two active ingredients are loaded on separate microparticles. The two types of microspheres are then mixed to obtain a formulation with the desired ratio of beta-receptor agonist and glucocorticosteroid, then administered simultaneously. Alternatively, the two types of microparticles are administered sequentially.

The microspheres comprising the active ingredient(s) are suspended in about 10 ml to about 20 ml of an appropriate physiologically acceptable liquid carrier. In some embodiments using separate microspheres of the active ingredients, the microspheres are mixed together in the liquid carrier. In other embodiments, each type of microspheres is separately mixed with a liquid carrier. In some embodiments, the microsphere suspension is then injected subcutaneously just below the dermis in 1.0 ml aliquots to cover about 2.0 cm² area per ml of the microsphere suspension, for example, for the treatment of cellulite. In some embodiments, about 10 to about 20 injections are administered to cover an area of from about 20 cm² to about 40 cm². Larger and/or smaller areas are treated in various embodiments. Alternatively, in some embodiments, bolus injections of 1.0 ml to 10.0 ml are injected into fat accumulations, such as the submental regions, lateral hips, and buttocks. Alternatively, injections as described above are made separately and sequentially in the same locations using two microsphere formulations encapsulating each active ingredient.

In some embodiments, needless injection is used to administer the microparticulate formulations as suspensions or as powdered loaded microparticles, i.e., without a liquid carrier.

PLGA 15 microspheres encapsulate hydrophobic compounds more readily than hydrophilic compounds. To increase loading of hydrophilic active ingredients, in some embodiments, the microspheres are modified with polyethylene glycol units, as discussed above. Microspheres of certain sizes are substantially not absorbed into the blood or removed by lymph, thereby providing localized release of the active ingredient(s) within a target region. For example, in some embodiments, the microspheres are about 20 μm to about 200 μm in diameter, e.g., about 30 μm to about 175 μm, about 50 μm to about 150 μm, about 75 μm to about 125 μm, or any other diameter from about 20 μm to about 200 μm. The size of the microsphere also affects the release profile of the active ingredient(s) in the tissue. In general, larger microspheres provide a longer and more uniform release profile. Accordingly, in some embodiments, the average particle size in the formulation will be selected based on the desired release duration.

In an exemplary embodiment, a sustained release formulation comprises about 0.5 mg to about 7.5 mg (e.g., about 0.7 mg, 1 mg, 1.5 mg, 2.0 mg, 2.5 mg, 3 mg, 3.5 mg, 4 mg, 4.5 mg, 5 mg, 5.5 mg, 6 mg, 6.5 mg, 7 mg, or any other amount from about 0.5 mg to about 7.5 mg) of salmeterol 1 and/or formoterol 2, and about 1.5 mgs to about 7.5 mgs (e.g., about 2 mg, 2.5 mg, 3 mg, 3.5 mg, 4 mg, 4.5 mg, 5 mg, 5.5 mg, 6 mg, 6.5 mg, 7 mg, or any other amount from about 1.5 mg to about 7.5 mg) of dexamethasone 6, fluticasone propionate 7, and/or budesonide 8 encapsulated in about 100 milligrams of polylactide glycolide (PLGA) 15 copolymer microspheres at a ratio of about 70 lactide:30 glycolide. The amount of each active ingredient in the sustained release formulation depends on the period of controlled/sustained release required (about 3 days to about 12 months, e.g., 4 days, 5 days, 7 days, 10 days, 1 month, 45 days, 2 months, 3 months, 6 months, 8 months, 9 months, or any other release period from about 3 days to about 12 months). In some embodiments, the copolymer ratio and active ingredient encapsulation deliver up to about 1.0 μg/day (e.g., about 0.02 μg/day, 0.04 μg/day, 0.06 μg/day, 0.07 μg/day, 0.1 μg/day, 0.2 μg/day, 0.4 μg/day, 0.5 μg/day, 0.6 μg/day, 0.8 μg/day, or any other amount from about 0.02 μg/day to about 1.0 μg/day) of salmeterol 1 and/or up to about 0.5 μg/day (e.g., about 0.02 μg/day, 0.04 μg/day, 0.06 μg/day, 0.07 μg/day, 0.08 μg/day, 0.09 μg/day, 0.1 μg/day, 0.2 μg/day, 0.3 μg/day, 0.4 μg/day, or any other amount from about 0.02 μg/day to about 0.5 μg/day) of formoterol, and up to 5 μg/day (e.g., about 0.2 μg/day, 0.4 μg/day, 0.5 μg/day, 0.7 μg/day, 0.9 μg/day, 1.0 μg/day, 1.5 μg/day, 2 μg/day, 2.5 μg/day, 3 μg/day, 3.5 μg/day, 4 μg/day, 4.5 μg/day, or any other amount from about 0.2 μg/day to about 5 μg/day) of fluticasone and/or budesonide 6 per about 1 mg of copolymer for up to about 30 days (e.g., about 1 day, 2 days, 3 days, 5 days, 7 days, 10 days, 14 days, 18 days, 21 days, 24 days, 27 days, or any other period up to about 30 days). In another exemplary embodiment, the sustained release formulation to be administered comprises methylprednisolone acetate as a crystalline microparticle suspension with a beta-2 adrenergic agonist. In various embodiments, the beta-2 adrenergic agonist is formulated in solubilized form, a crystalline microparticle suspension form, or both. In some embodiments, the sustained release formulation also comprises solubilized methylprednisolone succinate so as to provide immediate effects (due to rapid release) in addition to the sustained effect from the crystalline form of methylprednisolone acetate.

In some embodiments, the one or more beta-2 agonists and methylprednisolone are provided mixed together prior to administration (e.g., injection). In other embodiments, the one or more beta-2 agonists and methylprednisolone are mixed at the time of administration (e.g., by injection).

In some embodiments, a selected sustained corticosteroid release formulation (e.g., methylprednisolone crystalline suspension, methylprednisolone solution, or combination of crystalline suspension and solution) is delivered alone up to about 7 days prior to the beta-2 agonist (e.g., at least 12 hours, one day, 2 days, 3 days, 4 days, 5 days, 6 days, or any other time period from about 12 hours to about 7 days) to allow for beta adrenergic receptor upregulation.

In some embodiments, the subject to be treated is provided a non-sustained release formulation. In some embodiments, the non-sustained release formulation, after a single dose, provides activity of one or more long-acting selective beta-2 agonists for a duration from about 4 hours to about 24 hours, e.g., about 6 hours, 8 hours, 10 hours, 12 hours, 16 hours, 18 hours, 21 hours, or any other duration of beta-2 agonist activity from about four hours to about 24 hours.

In other embodiments, the subject to be treated is provided a non-sustained release formulation comprising short-acting selective beta-2 agonists, which have activities that last less than about four hours (e.g., about 3.5 hours, 3 hours, 2.5 hours, 2 hours, 1.5 hours, 1.3 hours, about 1 hour, 0.5 hours, or any other duration from less than about four hours to about 0.5 hour).

In an exemplary embodiment, a non-sustained release injectable formulation comprises from about 100 μg to about 250 μg of salmeterol xinafoate (e.g., about 105 μg, 110 μg, 125 μg, 150 μg, 175 μg, 190 μg, 200 μg, 210 μg, 225 μg, or any other amount of salmeterol xinafoate from about 100 μg to about 250 μg) and from about 500 μg to about 1000 μg of fluticasone propionate (e.g., about 600 μg, 650 μg, 700 μg, 730 μg, 740 μg, 800 μg, 825 μg, 875 μg, 900 μg, 930 μg, 950 μg, or any other amount of fluticasone propionate from about 500 μg to about 1000 μg) formulated in a volume of up to about 10 ml (e.g., about 0.1 ml, 0.3 ml, 0.5 ml, 0.7 ml, 1.1 ml, 1.5 ml, 2 ml, 2.5 ml, 3 ml, 3.5 ml, 4 ml, 5 ml, 6 ml, 7 ml, 8 ml, 9 ml, or any other volume from about 0.1 ml to about 10 ml) of an excipient compatible with administration into the orbit. In some embodiments, the excipient concentration is kept below 1% (e.g., about 0.05%, 0.2%, 0.3%, 0.4%, 0.5%, 0.6%, 0.8%, or any other concentration from about 0.05% to less than about 1%.

In some embodiments, formulations are delivered transdermally using any suitable method known in the art, for example, as a topically applied cream or through a patch. Alternatively, other transdermal delivery means known in the art are also useful, for example, electrical. In particular, long-acting beta-2 agonists, such as formoterol 2, salmeterol 1, or bambuterol 3, and glucocorticosteroids are suited for topical application to the skin due to their hydrophobicity. Sustained release embodiments of transdermally deliverable formulations are provided, for example, using a biodegradable, biocompatible active ingredient-polymer formulation or liposome formulation, as described herein.

In some embodiments, thyroid hormone is included in one of the foregoing formulations to up-regulate beta adrenergic receptors, or increase the beta adrenergic receptor number on the cell surface, and down-regulate alpha receptors, or decrease the alpha receptor number on the cell surface. In various embodiments, thyroxine (T4) 18, triiodothyronine (T3) 19, stereoisomers of these hormones, or any combination thereof are used in the for this purpose. Levothyroxine is a stereoisomer of thyroxine, which has a longer half life. In some embodiments, any of these thyroid hormones are combined with one or more beta adrenergic agonists and administered to a regional fat accumulation.

In some embodiments, a formulation for use in the methods described herein comprises a combination of the long-acting selective beta-2 agonists, salmeterol or formoterol and physiologic salts or solvates thereof. Without wishing to be bound by theory, it is believed that the combination of these compounds has an enhanced effect in improving the appearance of regional fat accumulations and cellulite. The hormones may also be combined with beta adrenergic agonists in a sustained release formulation, such as crystalline microparticle suspension, as described herein, to reduce the administration frequency. T3 and T4 may be combined with selective long-acting beta 2 agonists in crystalline microparticle suspension formulations for injection and sustained release in regional fat accumulations.

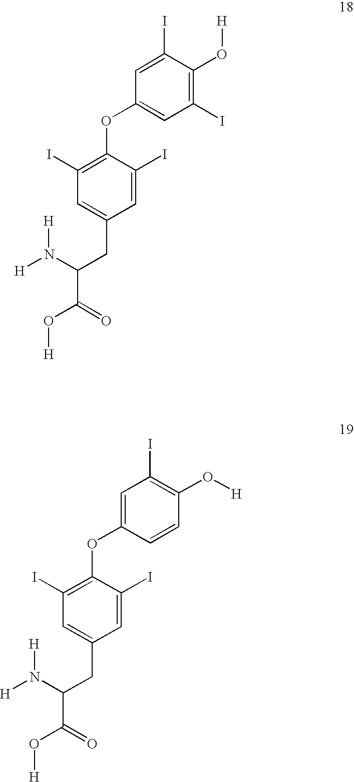

Atrial natriuretic peptide (ANP) is part of the natriuretic peptide family that has been demonstrated to induce lipolysis in human fat cells. Brain natriuretic peptide (BNP), C-type natriuretic peptide, and dendroaspis natriuretic peptide are also part of this family with ANP having the most potent lipolytic activity. Natriuretic peptides mediate their biological actions via specific receptors. The natriuretic peptide receptor A (NPrA) and NPrB possess an intrinsic guanyl cyclase activity and promote intracellular cGMP increase when stimulated. The NPrC serves as a scavenger receptor for natriuretic peptides. Adipose tissue expresses NPrA and also NPrC mRNAs, suggesting that natriuretic peptides have a functional role in the tissue. In human fat cells, ANP can activate hormone-sensitive lipase (HSL) through an increase in cGMP production and HSL phosphorylation. HSL then breaks down triglycerides into non-esterified fatty acids (NEFAs) and glycerol. In some embodiments, natriuretic peptides, in particular ANP and BNP are used to treat regional fat accumulations and improve the cosmetic appearance of these fat accumulations and cellulite. In some embodiments, natriuretic peptides are used in a sustained release formulation, as described herein to reduce administration frequency and improve the effect.

Beta-adrenergic receptors (B-ARs) are present in skin tissues. The beta 2 subtype adrenergic receptor (B2-AR) is the only receptor expressed on the membranes of the major skin cell types including keratinocytes, fibroblasts, and melanocytes. B2-ARs are also found in antigen presenting Langerhans cells of the skin. Aberrations in skin B2-AR receptors and function are associated with cutaneous disease. For example, patients with atopic eczema have a low density of B2-ARs on keratinocytes. In addition, psoriatic lesions are associated with reduced B2-AR numbers and with low cAMP levels (the main secondary messenger of B2-AR receptor activation) even after exposure to selective beta adrenergic agonists. B2-ARs are also found on white blood cells, lymphocytes and macrophages. These cells are also present in the skin in various inflammatory conditions such as atopic dermatitis and psoriasis.

Stimulation or inhibition of B2-AR can invoke various responses in these cells of the skin. For example, B2-AR stimulation with drugs can cause fibroblast proliferation and collagen production. In keratinocytes, B2-AR stimulation can inhibit proliferation, migration, and differentiation. Melanocytes will respond to B2-AR stimulation by increasing melanin production. Migration and antigen presentation by Langerhans cells is reduced upon exposure to B2-AR receptor agonists. Lymphocyte migration and proliferation is inhibited by B2-AR agonists. Activation of B2-AR receptors in dermal fibroblasts causes proliferation and increase fibroblast migration increases collagen production. In some embodiments, forskolin is used for the treatment of dermal conditions to produce the above-mentioned effects where defects in the B2-AR limit the activity of the long-acting beta adrenergic agonist.

In some embodiments, the above-mentioned drugs and combinations are used for treating immune and inflammation-related dermal conditions including psoriasis, atopic dermatitis, vitilligo, hypopigmentation, stria, and wrinkles or rhytids. In one embodiment, a combination of long-acting selective beta 2 agonists and ketotifen is administered. The principle of upregulating the beta adrenergic receptor number and reducing the desensitization of the beta adrenergic receptor through the use of ketotifen (or a glucocorticoid) in combination with a selective beta 2 agonist, such as salmeterol or formoterol is useful in the treatment of dermal conditions such as dermatitis (e.g. atopic) and psoriasis. Other drugs which stabilize the B2-AR and/or up regulate the receptor for treating dermal conditions include, but are not limited to, thyroid hormone, 1,25 dihydroxy vitamin D3 or it analogue, and bioflavanoids such as quercetin or fisetin. In some embodiments, darkening of skin or hypopigmented areas, such as occurs in vitilligo, stria, or a lack of sunlight is treated with the proposed combinations and principles of beta adrenergic receptor upregulation and/or stabilization as described herein. In one embodiment, a combination of long-acting beta adrenergic agonists with other drugs that stabilize and/or increase the B2-AR is used to treat the just-mentioned conditions. In some embodiments, a combination of long-acting beta adrenergic agonists with other drugs that enhance the cAMP response to B2-AR receptor stimulation is utilized. In one embodiment, a combination of formoterol with ketotifen is used to treat psoriasis. In some embodiments, where the B2-AR has defective activity, forskolin is used in the combination. In some embodiments, formoterol fumarate and ketotifen fumarate and or other physiologic salts or solvates of these drugs, or stereoisomers of ketotifen fumarate and formoterol fumarate or other physiologic salts or solvates of these drugs, are used for the treatment of psoriasis. Antigen presentation by the Langerhans cells may incite the immune component of the disease. The pathogenesis of psoriasis includes overproliferation of keratinocytes and immune inflammatory reactions, including lymphocyte (such as T-cells) migration and activation in the psoriatic lesion. Psoriasis may be characterized by T-helper 1 (Th1) type responses. In some embodiments, long-acting beta adrenergic agonists are used to control the proliferation of the keratinocyte and lymphocytes including T-Cells. In some embodiments, long-acting beta adrenergic agonists are also used to inhibit Th1 responses. In some embodiments, long-acting beta adrenergic agonist treatment is used to decrease Langerhans cell migration and antigen presentation. In some embodiments, Ketotifen can be used to stabilize and upregulate B2-ARs on lymphocytes, keratinocytes, or dermal fibroblasts. In addition ketotifen inhibits the release of cytokines such as tumor necrosis factor alpha (TNF-Alpha). TNF-Alpha plays a role in the pathogenesis of psoriasis. Thus, blocking the action of this cytokine (e.g., with antibodies), or reducing the release of the cytokine reduces the severity of skin lesions. In some embodiments, ketotifen is administered to inhibit T-Cell activity and thereby reduce inflammatory immune responses.

In another embodiment long-acting beta adrenergic agonists, such as formoterol fumarate, its stereoisomers, as physiologic salts or solvates thereof, are combined with 1,25-dihydroxy Vitamin D3 or its analogues. 1,25-dihydroxy Vitamin D3 can enhance beta-adrenergic adenylate cyclase responses in keratinocytes. In conditions, such as psoriasis, cAMP levels are low or cAMP formation is impaired. Accordingly, in some embodiments, where a subject is suffering from psoriasis Vitamin D3 is administered in combination with one or more long-acting beta adrenergic agonists.

In some embodiments, B2-AR agonists are administered for the treatment of skin wrinkles and skin stria, or stretch marks. Cutaneous stria are characterized by a thinning of the dermis, with a loss of collagen and hypopigmention. Long-acting beta adrenergic agonists promote the recruitment and proliferation and collagen production of dermal fibroblasts in the stria. In addition, they stimulate melanocytes to repigment the stria. Thus, in some embodiments one or more long-acting beta adrenergic agonists are used in combination with other drugs to stabilize and up regulate the B2-AR such as those disclosed above, including ketotifen, glucocorticoids, thyroid hormone, and bioflavanoids quercetin and fisetin. In some embodiments, forskolin is used with or without the long-acting beta adrenergic agonist and in combination with the previously disclosed compounds (e.g. Quercetin, fisetin, glucocorticoid, or ketotifen) to treat cutaneous stria and wrinkles.

In some embodiments, topical application of the drugs or drug combinations is utilized. In one embodiment, formoterol is the selected long-acting beta adrenergic agonist. For an individual substance the partition coefficient is generally measured as the Octanol:Water ratio or “Log P,” and is a measure of a given substance's relative affinity for Octanol vs. Water. The higher the Log P, the more a substance tends to be attracted to Octanol and vice versa. In other words, it provides a relative measure of lipophilicity versus hydrophilicity for a given substance. For delivery of agents into the skin an optimal Log P ranges from 1.0 to 5.0. Formoterol has a Log P in the range of 2-4. Ketotifen has similar physical properties that allow it to be delivered into and across the skin.

A variety of topical formulations, including ointments and creams, are suitable for delivery of the drugs or drug combinations to keratinocytes and dermal fibroblasts. Exemplary topical vehicles for the proposed combinations include, but are not limited to, terpenes (e.g. cineole, linalyl acetate, menthanone, d/l-menthol), fatty acid esters (e.g. isopropyl myristate, ethyl oleate, isopropyl palmitate, butyl myristate), and longer chain alcohols (1-octanol, 1-decanol, 1-dodecanol). N-methyl-pyrrolidone combined with terpenes, fatty acid esters, and longer chain alcohols. Ratios of terpenes, fatty acid esters, and longer chain alcohols to N-methyl-pyrrolidone may be from 100:0 up to a maximum 60:40 weight to weight. In some embodiments, needless intradermal injection of the drugs or drug combinations is used for treatment of wrinkles and other dermal conditions.

The combination of a selective long acting beta adrenergic agonist and ketotifen is also suitable for the treatment of cachexia. Formoterol and clenbuterol are beta adrenergic agonists with anabolic effects that increase muscle mass. Ketotifen can cause weight gain, which may be due to increased food intake secondary to appetite or satiety effects. Hence, the combination may have pronounced effects on cachexia, or wasting syndromes, secondary to other medical conditions such as HIV infection, cancer, or heart failure. Additionally, as previously disclosed the combination of formoterol or clenbuterol and ketotifen may have enhanced effects by increasing beta adrenergic receptor numbers and reducing receptor deactivation.

In some embodiments, B2-AR agonists are administered to increase skeletal muscle mass and cause hypertrophy and increased protein synthesis, effects which are mediated through intracellular in increases cAMP levels. Similar to adipocytes, exposure to B2-AR agonists results in receptor down regulation. Thus, the disclosed compositions, formulations, combinations can also be used for treating skeletal muscle injury or conditions where increasing skeletal muscle mass is important. In some embodiments, the methods described herein are used to increase facial muscle tone and provide a more youthful appearance. In some embodiments, the methods described herein are used to treat strabismus or lazy eye by strengthening ocular muscles. In some embodiments, the drugs and drug combinations described herein are provided in a sustained release carrier formulation. Specific combinations include long-acting beta adrenergic agonists, such as formoterol fumarate or salmeterol xinafoate, and compounds that reduce desensitization of B2-ARs, e.g., B2-ARstabilizers/upregulators glucocorticoids or ketotifen. In some embodiments, the combinations are formulated with a sustained release carrier such as PLGA microparticles. In some embodiments, bioflavanoids, such as quercetin or fisetin, are also used to decrease B2-AR desensitization. In some embodiments, glucocorticoids and long-acting beta adrenergic agonists are coadministered to a subject to repair a muscle injury. In some embodiments, agents that increase cAMP such as forskolin are used for treating skeletal muscle injury or improving facial muscle tone.

In some embodiments, the disclosed compositions, formulations (e.g., sustained release formulations), and combinations described herein are used for treating visceral fat accumulation commonly associated with obesity related comorbidities such as diabetes and hypertension. In some embodiments, drug or drug combination formulations are delivered to a visceral fat accumulation by using an endovascular catheter to cannulate the feeding artery or arteries of the visceral fat pad. In some embodiments, cannulation is performed by obtaining vascular access in the femoral artery, maneuvering a guide wire to the celiac trunk artery, and then distally into the gastric artery of the greater curvatures. This artery has multiple feeding arteries to the regional omental fat accumulation. A hollow delivery catheter is then positioned in the artery over the guidewire allowing the administration of the drug or drug combinations or sustained release formulations of the drugs or drug combinations as previously disclosed. In some embodiments microparticles for delivery are manufactured to cross the capillary membrane for deposition in the tissue. In some embodiments, the catheter is maneuvered into the superior and inferior mesenteric arteries to deliver drug, drug combinations, or sustained release formulations of drugs and drug combinations to the mesenteric regional fat accumulations.

In some embodiments, inducing lipolysis and inhibiting fat cell growth in regional fat accumulations, whether visceral or subcutaneous, have additional health benefits through the shrinkage of the average fat cell diameter or volume. Large volume fat cells actively secrete pro-inflammatory and deleterious hormones such as TNF-alpha and interleukins (“adipokines”), which contribute to comorbidities associated with fat, such as diabetes. By reducing the size of these fat cells and therefore the deleterious adipokine secretion, improvements in fat-related comorbidities are realized.

In some embodiments, the disclosed compositions, formulations (e.g., sustained release formulations), and combinations described herein are used for treating obstructive sleep apnea. Obstructive sleep apnea occurs when the airway is temporarily blocked during sleep, leading to hypoxia, high blood pressure, cardiac dysrhythmia, and a higher risk of death. Excessive fat in the pharynx and soft palate it believed to contribute to this blockage. Obese people have a higher incidence of sleep disorders and persons with sleep apnea have excessive fat in the palate and pharynx on MRI. Thus, in some embodiments, formulations described are administered to a subject to reduce the symptoms of sleep apnea. In some embodiments, the formulations are administered locally (e.g., by injection) into the palate or pharynx transorally. In some embodiments, the formulations are administered by subcutaneous into the region the neck to reduce the obstructive symptoms

While certain embodiments have been described, these embodiments have been presented by way of example only, and are not intended to limit the scope of the disclosure. The formulations, methods, and systems described herein may be embodied in a variety of other forms. Furthermore, various omissions, substitutions and changes in the form of the formulations, methods, and systems described herein may be made without departing from the spirit of this disclosure. The accompanying claims and their equivalents are intended to cover such forms or modifications.

### EXAMPLES

The following specific examples are to be construed as merely illustrative, and not limitative of the remainder of the disclosure in any way whatsoever.

### Example 1

### In Vitro Lipolysis Assay of Adipocytes by Beta Adrenergic Agonists and Glucocorticosteroids

In the in vitro lipolysis assay, glycerol was detected in cell culture media via a spectrophotometric measurement after chemical oxidation with hydrogen peroxide. Glycerol was measured over a three hour time period. Levels of lipolysis in cultured human adipocytes were tested after exposure to a beta adrenergic agonist alone, a glucocorticosteroid alone, or the combination of the two for one or more preincubation periods as described in more detail below.

Isolation of Pre-Adipocytes and Differentiation into Adipocytes:

Human subcutaneous adipocytes were used in the lipolysis assay. Adipose tissue was harvested from liposuction or lipectomy and pre-adipocytes were isolated as follows. Briefly, fat tissue was minced and incubated at 37° C. in Krebs-Ringer bicarbonate buffer containing 1% bovine serum albumin and 0.1% collagenase in an oxygen-rich shaking chamber (5% CO₂; 75 strokes/min) for 1 hour. The suspension was filtered through a 400 μm nylon mesh and centrifuged for 1 min at 100 g. The pre-adipocytes in the supernatant were washed twice with and then plated in 96 well plates at a density of cells/well. The pre-adipocytes were cultured in maintenance medium for seven days as they differentiated into adipocytes.

### Reagents

Wash Buffer (Krebs Ringer Buffer (KRB) without serum; [Sigma, K4002-10X1L]) —stored at 4° C.
Assay Buffer (KRB with 1% FBS; [FBS from Gibco, 26140-079]) —stored at 4° C.
Maintenance Medium stored at 4° C.
Glycerol Reagent A (Zen-Bio, RGTL-15 or RGTL-40) —after reconstituting, store at 4° C. protected from light.
Glycerol stock solution (1 M), prepared by diluting glycerol [Sigma G2025-500 ML] in Wash Buffer (no serum) —stored at −20° C.

### Lipolysis Assay:

At −21 hours before the lipolysis assay, medium was removed from each well and replaced with 75 μl of Maintenance Medium containing appropriate drug or DMSO (vehicle) concentrations (see Experimental Design section below). Each test drug or control treatment was applied to 8 wells/group (12 treatment groups per 96-well plate). At −3 hours prior to the lipolysis assay, each well was washed two times with Wash Buffer (200 μl/wash), filled with test or control solutions made up in Assay Buffer (75 μl/well), and then incubated for three hours, i.e., until measurement of glycerol content in the Assay Buffer. For some groups, a drug was only added for the three hour incubation period (see Experimental and Control Groups Below). One hour prior to the assay, seven glycerol standards ranging from 200 μM to 3.125 mM were prepared by serial dilution in Assay Buffer.

The glycerol content of the Assay Buffer from each well following the incubation was used as an index for lipolysis, where an increase in glycerol indicated lipolysis. Glycerol levels were assayed colorimetrically via a commercial glycerol assay kit (Randox Laboratory, United Kingdom) and quantified by comparison to a glycerol serial dilution standard curve (3 μM-200 μM). Glycerol concentrations for each well were normalized to cell density.

### Experimental Design:

In each of the following control or experimental groups n=8 corresponding to the glycerol measurements for 8 wells from a 96-well cell culture plate.

TABLE 1
Summary of In Vitro Lipolysis Assay Experimental Design

| | | | |
|---|---|---|---|
| | | | Results |
| | | | shown |
| | | | in |
| Group(s) | 18 hour Incubation | 3 Hour Incubation | FIG. |

Experiment 1

| | | | |
|---|---|---|---|
| 1 (negative | 0.1% DMSO | 0.1% DMSO | 2* |
| control) | | | |
| 2-11 | 0.1% DMSO | Formoterol (10⁻¹³ M-10⁻⁴M) | 2 |
| 12 (positive | 0.1% DMSO | Isoproterenol 10⁻⁶ M | 2 |
| control) | | | |

Experiment 2

| | | | |
|---|---|---|---|
| 13 (negative | 0.1% DMSO | 0.1% DMSO | 3* |
| control) | | | |
| 14-18 | 0.1% DMSO | Salmeterol (10⁻⁸ M-10⁻⁴ M) | 3 |
| 19 (positive | 0.1% DMSO | Isoproterenol 10⁻⁶ M | 3 |
| control) | | | |

Experiment 3

| | | | |
|---|---|---|---|
| 20 (negative | 0.1% DMSO | 0.1% DMSO | 4* |
| control) | | | |
| 21-22 | 0.1% DMSO | Budesonide 10⁻¹⁰ M and 10⁻⁷ M | 4 |
| 23-26 | Budesonide (10⁻¹² M-10⁻⁶ M) | Budesonide (10⁻¹² M-10⁻⁶ M) | 4 |
| 27 (positive | 0.1% DMSO | Isoproterenol 10⁻⁶ M | 4 |
| control) | | | |

Experiment 4

| | | | |
|---|---|---|---|
| 28 (negative | 0.1% DMSO | 0.1% DMSO | 5* |
| control) | | | |
| 29 | Salmeterol 10⁻⁶ M | Salmeterol 10⁻⁶ M | 5 |
| 30 | Salmeterol 10⁻⁶ M + | Salmeterol 10⁻⁶ M + | 5 |
| | Budesonide 10⁻⁶ M | Budesonide 10⁻⁶ M | |
| 31 | Salmeterol 10⁻⁸ M | Salmeterol 10⁻⁸ M | 5 |
| 32 | Salmeterol 10⁻⁸ M + | Salmeterol 10⁻⁸ M + | 5 |
| | Budesonide 10⁻⁶ M | Budesonide 10⁻⁶ M | |
| 33 (positive | Isoproterenol 10⁻⁶ M | Isoproterenol 10⁻⁶ M | 5 |
| control) | | | |

*All groups are plotted as fold or % difference relative to negative control group data.

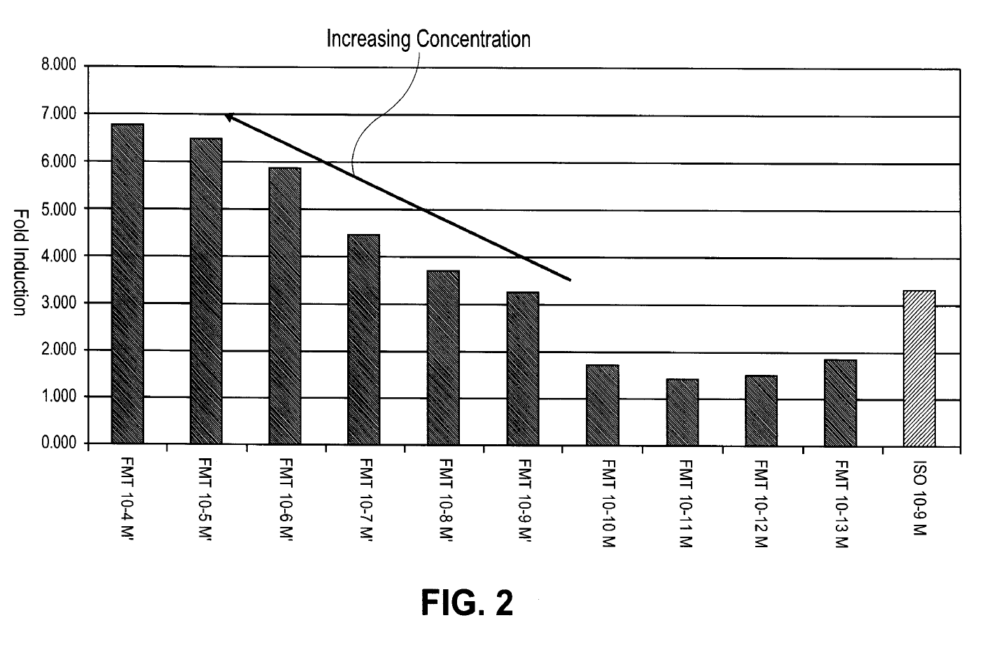

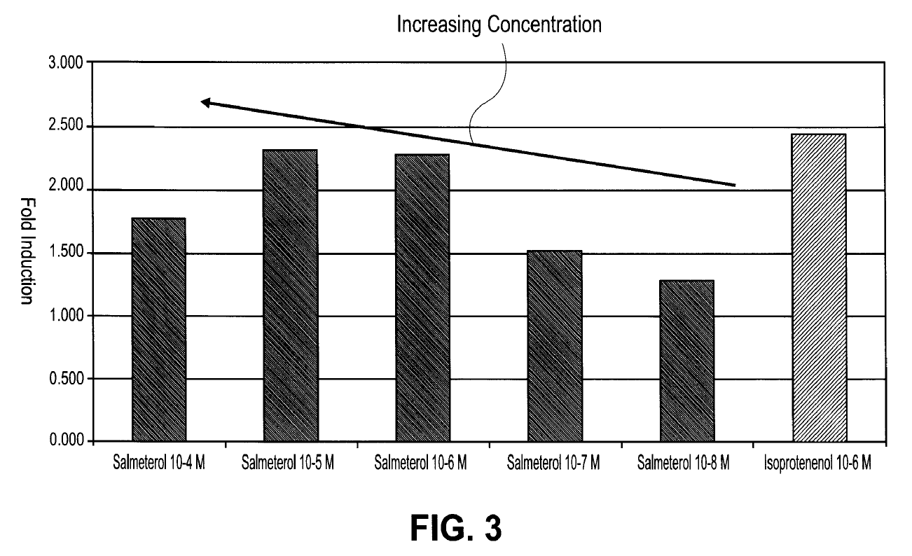

As shown in FIG. 2, the long-acting beta-2 adrenergic receptor agonist formoterol induced a dose-dependent increase in lipolysis of greater than six fold after a three hour incubation, which, for concentrations of 10⁻⁶M and above, was greater than that observed for isoproterenol. Likewise, the long-acting beta-2 adrenergic receptor agonist salmeterol also induced a dose-dependent increase in lipolysis after a three hour incubation (FIG. 3), although the effect (slightly greater than two fold increase in lipolysis) was not as strong as that observed for Formoterol. Salmeterol-induced lipolysis was equal to or less than that observed for Isoproterenol.

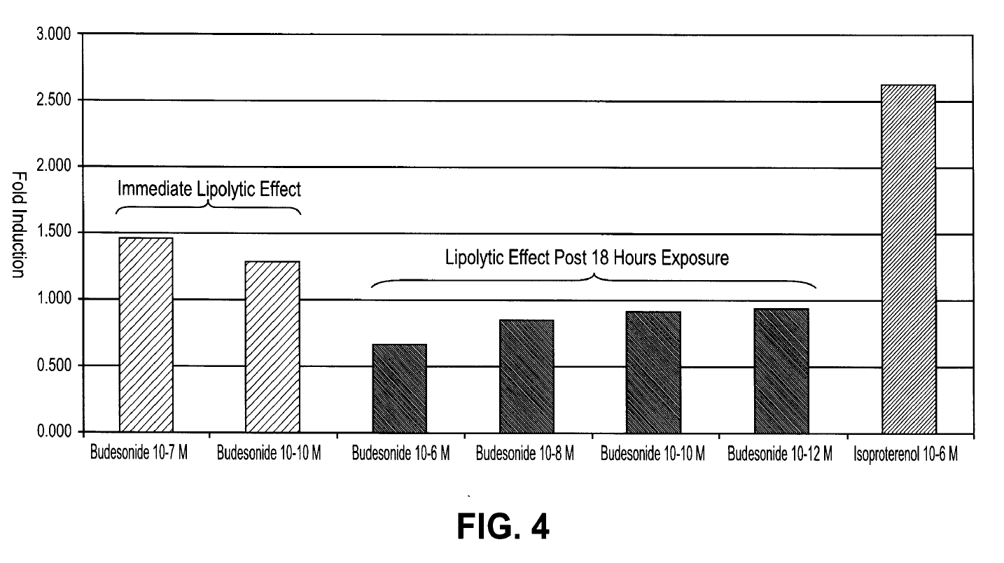

As shown in FIG. 4, the glucocorticosteroid budesonide induced a slight increase (up to about 1.5 fold) in lipolysis after three hours, which was lower than that observed for Isoproterenol (about 2.5 fold). In contrast, incubation with Budesonide alone for 18 hours actually caused a slight suppression of lipolysis in vitro.

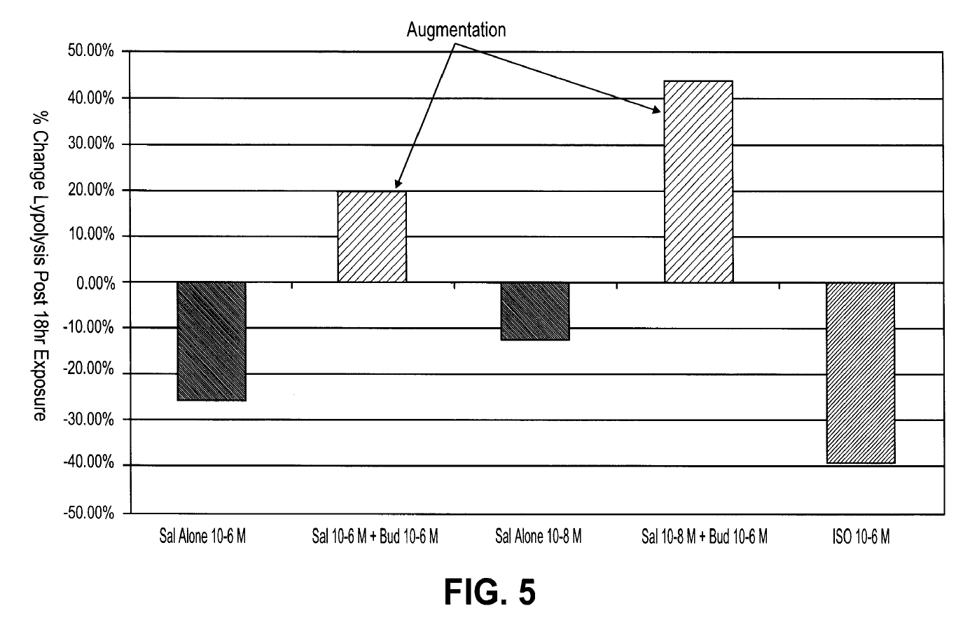

Incubation of adipocytes with Salmeterol (10⁻⁶M) for 18 hours decreased lipolysis (FIG. 4). Similarly, treatment with Isoproterenol for 18 hours resulted in decreased lipolysis. However, when Salmeterol was combined with Budesonide for an 18 hour incubation period, an increase in lipolysis was observed (FIG. 5).

Based on these data, we concluded that formoterol and salmeterol effectively induce lipolysis in cultured adipocytes over a period of three hours. However, Salmeterol actually decreases lipolysis when used for 18 hours, likely due to receptor desensitization or downregulation. Further, in the presence of the glucocorticosteroid Budesonide, Salmeterol is able to induce lipolysis even after 18 hours. Thus, Budesonide can be used to maintain or restore the ability of a beta-2 adrenergic agonist to induce lipolysis over long periods of time, likely by preventing down-regulation of beta-2 adrenergic receptors.

### Example 2

### Adipogenesis Inhibition by Beta Adrenergic Agonists and Glucocorticosteroids

A non-limiting example of an assay for inhibition of adipogenesis is as follows:

### Cell Culture:

3T3-L1 preadipocyte cell line (ATCC, Manassas, Va.) are plated at 4×10⁵ cells per T75 ml flask in Dulbecco's Modified Eagle's Medium (DMEM) with 10% normal calf serum and 1% penicillin/streptomycin antibiotics. Cells are incubated at 37° C., 5% CO₂. After three days, cells are detached by trypsin, counted and resuspended into 24 well plates with 6×10⁵ cells per well in 2 ml medium. After 1-2 days, cells are near-confluence and ready for adipogenesis.

### Adipogenesis Materials:

### Adipogenesis Initiation Medium: DMEM/10% Fetal Bovine Serum/0.5 mM IBMX/1 μM Dexamethasone

Adipogenesis Progression Medium: DMEM/10% Fetal Bovine Serum/10 μg/mL Insulin

### Adipogenesis Maintenance Medium: DMEM/10% Fetal Bovine Serum

### Negative Control Medium: DMEM/10% Normal Calf Serum

### Adipogenesis Protocol:

1.8 ml of medium is removed from the wells and 2 ml Adipogenesis Initiation Medium added per well. Plates are incubated 48 hours at 37° C., 5% CO₂. 2 ml of medium is removed and 2 ml of Adipogenesis Progression Medium added per well. Plates are incubated 48 hours at 37° C., 5% CO₂. 2 ml of medium is removed and 2 ml of Adipogenesis Maintenance Medium added per well. Plates are incubated for at least 48 hours at 37° C., 5% CO₂. Intracellular lipid droplets accumulate in the cells for at least 5 days.

### Experimental Design:

Prior to adipogenesis, 3T3-L1 preadipocyte cells are pretreated at different stages with a beta 2 agonist and/or glucocorticosteroid. 24 h before addition of Adipogenesis Initiation Medium, cells are treated with the following:

Group 1: No Treatment

Group 2: 10⁻¹⁰ M Salmeterol

Group 3: 10⁻⁸ M Salmeterol

Group 4: 10⁻⁶ M Salmeterol

Group 5: 10⁻⁴ M Salmeterol

Group 6: 10⁻¹⁰ M Salmeterol+10⁻⁶M Budesonide

Group 7: 10⁻⁸M Salmeterol+10⁻⁶ M Budesonide

Group 8: 10⁻⁶M Salmeterol+10⁻⁶ M Budesonide

Group 9: 10⁻⁴ M Salmeterol+10⁻⁶ M Budesonide

Group 10: 10⁻⁶M Budesonide

Group 11: 10⁻¹⁰ M Budesonide

Group 12: 10⁻⁶ M Capsaicin, a known adipogenesis inhibitor

Another set of cells is treated with the above group 24 hours prior to addition of Adipogenesis Progression Medium and yet another set is treated 24 hours prior to Adipogenesis Maintenance Medium. In the control set, the cells are treated with the above group 24 hours prior addition of Negative Control Medium. Two other sets of 12 groups substitute salmeterol for the long-acting beta 2 agonist, formoterol, in one set and a short acting beta 2 agonist, albuterol, in the other set.

### Visualization of Intracellular Lipids:

Cells are harvested 5 days after addition of Adipogenesis Maintenance Medium. Cell medium is removed and plates are washed twice with phosphate buffered saline (PBS). 0.5 ml of Oil Red 0 solution (0.36% Oil Red O in 60% isopropanol) is added per well and plates are incubated at 15 minutes at room temperature. Staining solution is removed and wells are washed three times with 60% isopropanol. Stained plates are then photographed and/or scanned for visual analysis. Lipids are stained red.

### Lipid Quantification:

0.25 ml Dye Extraction Solution (CHEMICON International) is added to the stained wells. Plates are set on an orbital shaker or rocker for 15-30 min. The solution with the extracted dye is transferred to a cuvette and the absorbance read by a spectrophotometer at 520 nm.

### Example 3

### Beta-2 Agonists in Combination with Glucocorticosteroids Decrease Epididymal Fat Pad Mass

We sought to determine if a glucocorticosteroid reduces fat in vivo in a manner consistent with our in vitro lipolysis data as described in Example 1. To this end, we measured epididymal fat pad mass in rats treated with the long-acting beta-2 adrenergic agonist Formoterol alone and in combination with budesonide.

Male Sprague Dawley rats (˜500 g) were anesthetized under 4% isoflurane using a Matrix 3000 vaporizer. The animals, as listed in Table 2 below, were then injected 5 mm anterior to the posterior end of the fat pad with 0.4 ml of vehicle (2% PEG); Formoterol (3.48 μg/ml; dose=1.39 μg) in the vehicle; or Formoterol (3.48 μg/ml) plus Budesonide (10 μg/ml; dose=1.39 μg Formoterol and 4 μg Budesonide) in the vehicle. Each animal received a drug treatment on one side and vehicle (2% PEG) treatment on the contralateral side; each group was right-left counterbalanced with respect to drug and vehicle (see Table 2).

TABLE 2
Experimental Design of In Vivo Lipolysis Assay

| Group | Animal ID | Right epididymal fat pad | Left epididymal fat pad |
|---|---|---|---|
| 1 | 1 | Formoterol | Vehicle |
| | 2 | Formoterol | Vehicle |
| | 3 | Vehicle | Formoterol |
| | 4 | Vehicle | Formoterol |
| 2 | 5 | Formoterol :: Budesonide | Vehicle |
| | 6 | Formoterol :: Budesonide | Vehicle |
| | 7 | Vehicle | Formoterol :: Budesonide |
| | 8 | Vehicle | Formoterol :: Budesonide |

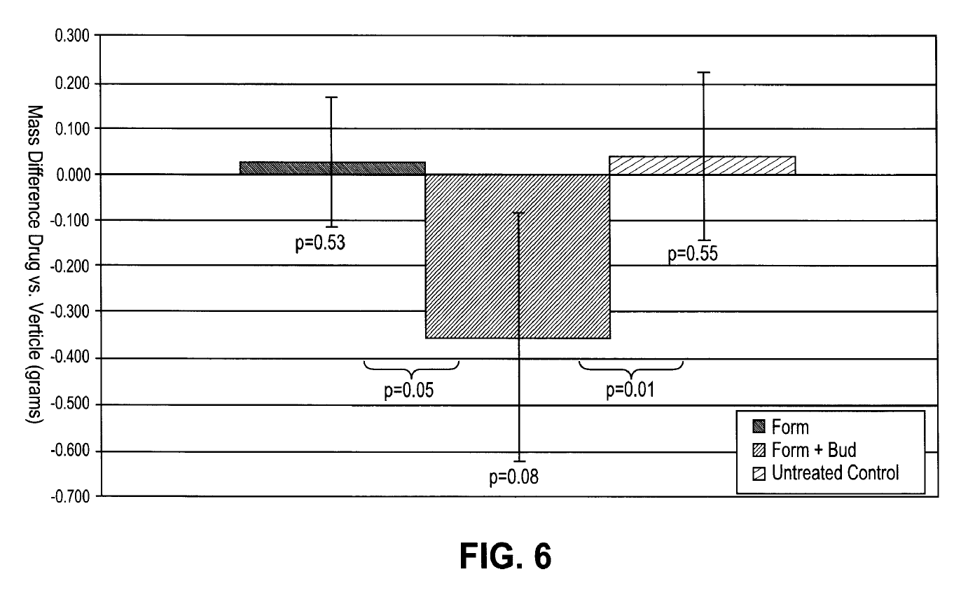

The injections were repeated at 24 and 48 hrs later, for a total of three injections. Twenty four hours after the final injection, animals were euthanized by an i.p.-injected overdose of pentobarbital (150 mg/kg), and the left and right epididymal fat pads from each animal were harvested and weighed (results shown in Table 3 and FIG. 6). Paired t-test and standard t-test was used for statistical analysis.

TABLE 3
Fat Pad Weight Δ (drug-vehicle)

Group

| | 1 | 2 |
|---|---|---|
| Animal 1 & 5 | −0.036 | −0.599 |
| Animal 2 & 6 | −0.138 | −0.115 |
| Animal 3 & 7 | 0.118 | −0.574 |
| Animal 4 & 8 | 0.166 | −0.124 |
| Mean | 0.028 | −0.353 |
| SD | 0.140 | 0.270 |

The Formoterol alone and Formoterol+budesonide treatment data showed in Table 3 were analyzed with a paired Student t-test, the results of which are shown in Table 4.

TABLE 4
Statistical Analysis of Fat Pad Weight Δ (drug-
vehicle) following Formoterol alone or Formoterol +
Budesonide Treatment

| Groups | Two-tailed p value |
|---|---|
| Group 1 (Formoterol) | 0.63 |
| vs | |
| vehicle | |
| Group 2 | 0.0792 |
| (Formoterol + Budesonide) | |
| vs | |
| vehicle | |

As shown in Table 4, Group 1 animals (treated with Formoterol alone) showed differences and variability consistent with naïve untreated control. The mean treatment effect for Formoterol alone was +0.028 g±0.140 g. Statistical analysis yielded a p value of 0.72 consistent with no trend toward a treatment effect. On the other hand, Group 2 animals (treated with Formoterol+Budesonide) showed a trend toward a significant treatment effect. The mean treatment effect was −0.353±0.270 with a p value=0.07.

We also performed a statistical analysis (Student t-test) for significant differences between the effect of the single and combination drug treatments, and also for a significant difference between the combination treatment versus untreated control animals. The results of these statistical analyses are shown in Table 5.

TABLE 5
Statistical Analysis of Differences in Effects Formoterol Alone
versus Formoterol + Budesonide; and Formoterol +
Budesonide versus Untreated

| Comparison (Mean difference in Epi fat | Two-tailed |
|---|---|
| pad weight) | P value |
| Group 2 (Formoterol + Budesonide) | 0.05 |
| vs | |
| Group 1 (Formoterol alone) | |
| Group 2 | 0.0131 |
| vs. | |
| untreated | |

As shown in Table 5 and FIG. 6, there was a trend towards a significant difference between Group 2 and Group 1 (p=0.05). There was a significant difference in the mean fat pad mass reduction in Group 2 versus untreated animals with a p=0.013.

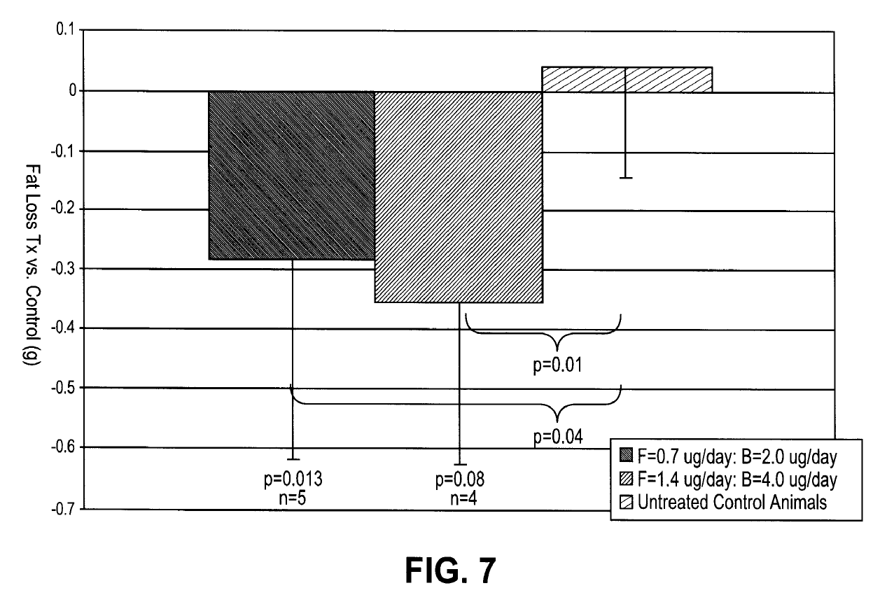

In a follow-up experiment, we examined the ability of formoterol plus budesonide to reduce fat pad mass in the above-described assay. As shown in FIG. 7, administering a dose combination of 1.4 μg formoterol+4.0 μm budesonide every other day resulted in a significant difference in mean epididymal mass difference in the treated versus untreated group (p=0.01). Likewise, even at a lower dose combination (0.7 μg/formoterol+2.0 μg/budesonide), the difference between the combination-treated and untreated control animals was significant (p=0.04).

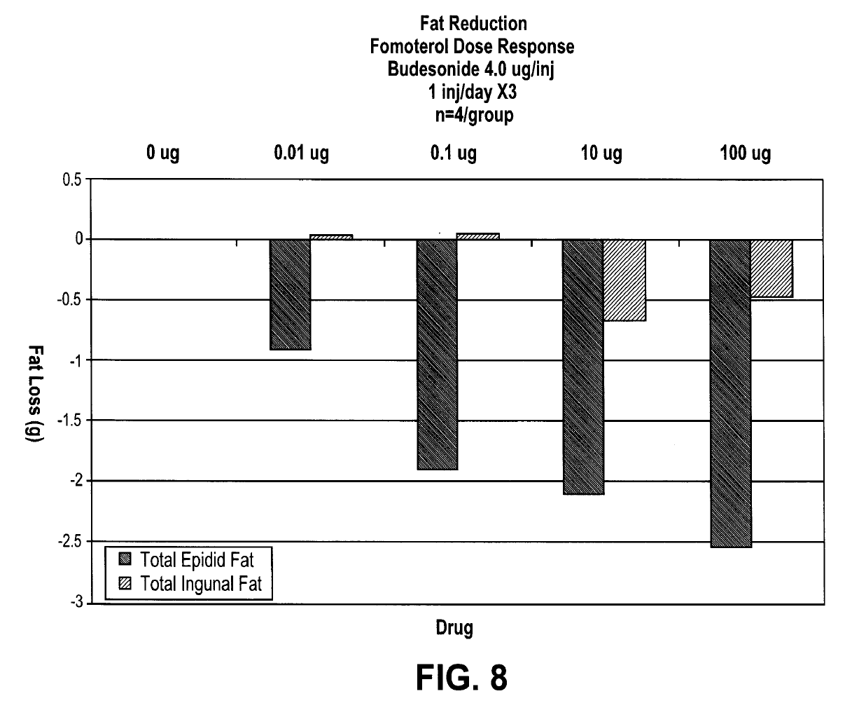

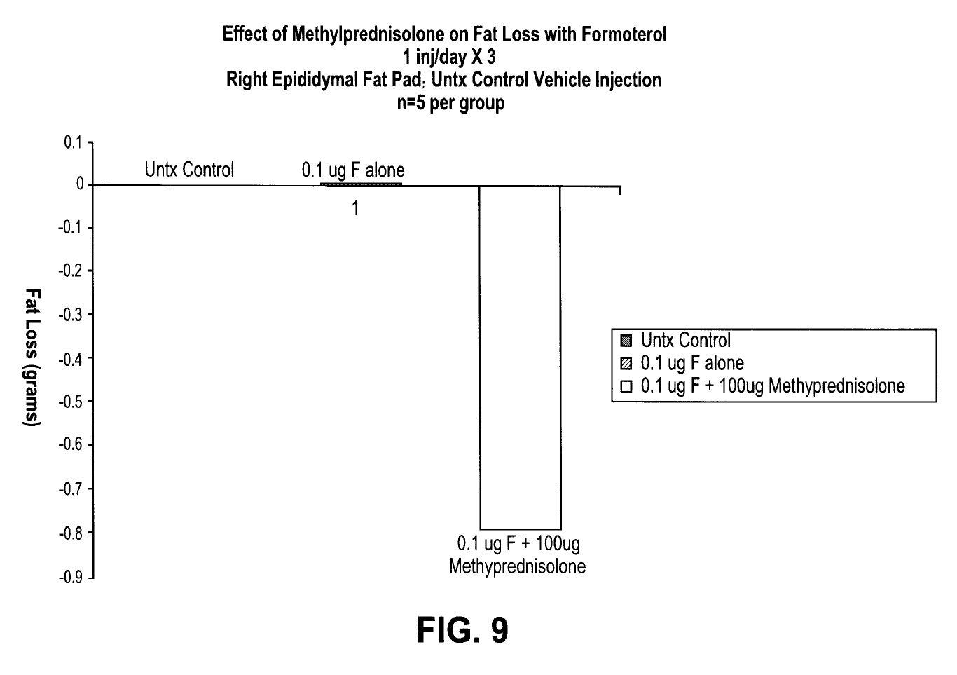

Finally, we performed a dose-response study by co-injecting into epididymal fat pads a range of doses of formoterol (0.01 μg to 100 μg) along with a constant dose of budesonide (4.0 μg). We then determined changes in epididymal and inguinal fat pad mass. As shown in FIG. 8, there was a dose-dependent reduction in epididymal fat pad mass. Further, as the dose of formoterol was decreased, the effect on inguinal fat pad loss was eliminated while the epididymal fat loss persisted. This result indicated that, as expected, the effect of the treatment in the epididymal fat pad was local to where the injection of formoterol and budesonide had been administered. In another experiment, we substituted budesonide with an approximately equipotent dose of methylprednisolone (100 μg). We fixed the dose of formoterol at 0.1 μg and tested a dose of formoterol alone. Right epididymal fat pads were injected once per day for three days. In the combination group, there was an average of 0.8 grams of epididymal fat loss relative to a vehicle-injected control group and essentially no fat loss in the formoterol only treatment group (FIG. 9).

Based on these data and their analysis, we concluded that a combination of a long-acting beta-2 agonist (e.g., Formoterol) and a glucocorticosteroid (e.g., budesonide) are likely to be effective for inducing local lipolysis and fat reduction in vivo.

### Example 4

### Beta-2 Agonists in Combination with Glucocorticosteroids Decrease Inguinal Fat Pad Mass

Studies were conducted in rats to determine the effect of the combination of the long-acting selective 2 beta adrenergic agonist salmeterol in combination with fluticasone propionate on the inguinal subcutaneous fat pad. Stock solutions of salmeterol and fluticasone in propylene glycol were diluted 1:20 with normal sterile saline to give approximately 1.0 μg of salmeterol and approximately 2.0 μg of fluticasone per ml of formulation. The active drug formulation was injected into the right inguinal fat pad using a needless injection device. Control formulation consisting of a 1:20 dilution of propylene glycol was injected into the left inguinal fat pad of the animal. Dose Group 1 animals (n=6) were injected every third day for 2 weeks. Dose Group 2 animals (n=6) were injected every other day for 1 week. At the end of the treatment period the fat pads were harvested and weighed to compare the mass of the right fat pad versus the left. Samples from each fat pad were taken for analysis of fat cell number and fat cell mean diameter.

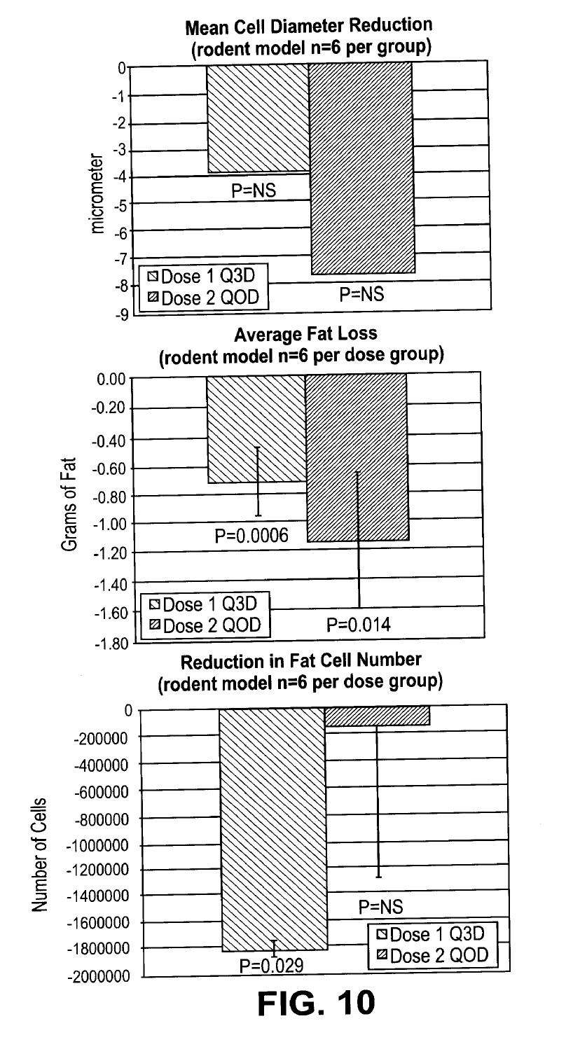

For Dose Group 2 animals the average fat pad mass of the treated side was 5.08 grams versus 6.2 grams for the control side. This yielded a statistically significant (p=0.014) fat loss of 1.12 grams+/−0.46 grams (mean+/−s.d.) of fat relative to the control side (FIG. 10). For Dose Group 1 the average fat pad mass of the treated side was 5.43 grams and the average fat pad mass of the control side was 6.14 g. This yielded a statistically significant (p=0.0006) fat loss of 0.72 grams of fat+/−0.23 grams (mean+/−s.d.) (FIG. 10). Samples were processed for cell counting and cell size analysis using a coulter counter. The mean cell diameter for Dose Group 2 animals was 7.64 micrometers less for the treated side versus the control (FIG. 10). The mean cell diameter for Dose Group 1 animals was 3.8 micrometers less for the treated versus the control (FIG. 10). A statistically significant (p=0.029) reduction in fat cell number averaging 1,840,000 cells was observed in Dose Group 1 animals after the 2 week treatment period (FIG. 10). This represented approximately 34% reduction in fat cell number of the analyzed sample. This data is particularly surprising and impressive when one notes that rat adipose tissue does not express beta 2 adrenergic receptors.

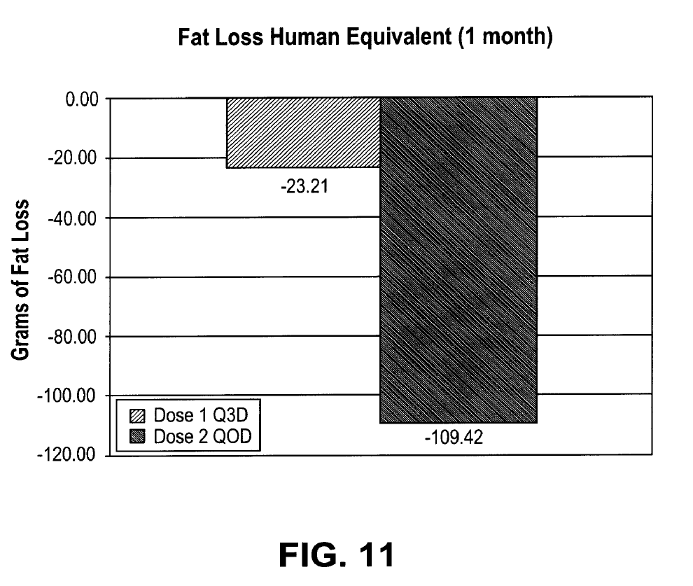

Rates of fat loss from the rodent studies were 0.036 grams of fat/day/μg of drug for Dose Group 2 and 0.008 grams of fat/day/μg of drug for Dose Group 1. Human equivalent dosing would be 100 μg per injection of salmeterol, which would yield a rate of 3.6 grams of fat/day at the Dose Group 2 every other day dosing regimen. As previously disclosed, the formulation is ideally suited for incorporation into a sustained release version to provide up to 1 month of drug release. In a sustained release version, at a human equivalent dosing, a single injection is projected to produce up to 109 grams (121 milliliters) of regional fat loss (FIG. 11). This human equivalent dosing outlined above is one half the maximal dose approved by the FDA for this formulation, so even larger amounts of fat reduction (perhaps 2 times) will likely be achieved through a daily dose equivalent. Fat reductions of this volume amount may be suitable for treating the regional fat accumulations and improving the appearance of fat.

The above data further support our conclusion that a combination of a long-acting beta-2 agonist (e.g., salmeterol) and a glucocorticosteroid (e.g., fluticasone propionate) are likely to be effective for inducing lipolysis and fat reduction in vivo.

### Example 5

### Clinical Testing for Treatment of Regional Fat Deposits with Compositions Comprising Beta Adrenergic Agonist and Glucocorticosteroid

A non-limiting example of such a clinical testing for treatment of regional fat deposits is as follows:

### Patient Selection:

Patients are to be 18 years of age or above and have no hypersensitivity to the administered drugs. They are diagnosed as having a lipoma or other undesirable regional fat deposit. All studies are to be performed with institutional ethics committee approval and patient consent.

### Study Design:

Test 1: This is a multicenter, dose escalation study of the combination therapy of salmeterol, a long-acting beta 2 agonist with the budesonide, a glucocorticosteroid. Patients receive an injection administration of a parenteral composition of the drug daily. Patients who do not achieve a reduction in fat mass or lipoma diameter after 1-3 weeks of therapy will receive an additional 1-3 weeks of therapy at a higher dose than what is originally assigned. Cohorts of 3-6 patients receive escalating doses of the combination drug until the maximum tolerated dose (MTD) is determined. The MTD is defined as the dose preceding that at which 2 of 3 or 2 of 6 patients experience dose-limiting toxicity.
Test 2: This is a randomized, multicenter study. The study length is 60 days. Patients are randomized to 1 of 18 treatment groups. For group 1, patients are given salmeterol alone daily at MTD. For group 2, patients are given budesonide alone daily at MTD. For group 3, patients are given salmeterol and budesonide concurrently at MTD. For Group 4, patients are given salmeterol daily, and budesonide on every other day. For Group 5, patients are given budesonide daily, and salmeterol on every other day. For Group 6, patients are given budesonide on every odd day and salmeterol on every even day. Groups 7-12 have the same dosing regime as 1-6 except the dosage is at one-fourth MTD. Groups 13-18 also have the same dosing regime as 1-6 except the dosage is at one-tenth MTD. In addition to the treatment groups, a control group is left untreated.

### Endpoint Assessment:

Patients are assessed for reduction of regional fat deposit mass at the conclusion of the study. A reduction of fat mass of at least 10% as measured by MRI or ultrasound within 60 days indicates a positive outcome.

### Example 6

### Pharmaceutical Compositions

Parenteral Compositions

### Example 6A

To prepare a parenteral pharmaceutical composition suitable for administration by injection, about 10 to 100 μg of a water-soluble salt of formoterol or salmeterol and about 40 μg to 400 μg of budesonide is dissolved in PEG and then mixed with 0.9% sterile saline to a final volume of 10 ml. The mixture is incorporated into a dosage unit form suitable for administration by injection.

### Example 6B

To prepare a parenteral pharmaceutical composition suitable for administration by injection, about 10 to 100 μg of a water-soluble salt of formoterol or salmeterol and about 100 μg to about 500 μg of methylprednisolone succinate is dissolved in buffered 0.9% sterile saline to a final volume of 10 ml. The resulting mixture is incorporated into a dosage unit form suitable for administration by injection.

### Example 6C

To prepare a parenteral pharmaceutical composition suitable for administration by injection, about 10 μg to about 100 μg of crystalline microparticles of a salt of formoterol or salmeterol is suspended in 10 ml of PEG and buffered sterile 0.9% saline. The mixture is incorporated into a dosage unit form suitable for administration by injection.

### Example 6D

To prepare a parenteral pharmaceutical composition suitable for administration by injection, about 100 μg to about 500 μg of methylprednisolone acetate or budesonide crystalline microparticles are suspended in 10 ml of PEG and buffered sterile 0.9% saline. The resulting mixture is incorporated into a dosage unit form suitable for administration by injection.

### Example 6E

To prepare a parenteral pharmaceutical composition suitable for administration by injection, about 10 μg to about 100 μg of crystalline microparticles of a salt of formoterol or salmeterol, and about 100 μg to about 500 μg of budesonide or methylprednisolone acetate crystalline microparticles are suspended in 10 ml of PEG and buffered sterile 0.9% saline. The resulting mixture is incorporated into a dosage unit form suitable for administration by injection.

Topical Gel Compositions

### Example 6F

To prepare a pharmaceutical topical gel composition, about 100 μg of salmeterol and about 100 μg of prednisolone is mixed with 1.75 g of hydroxypropyl cellulose, 10 ml of propylene glycol, 10 ml of isopropyl myristate and 100 ml of purified alcohol USP. The resulting gel mixture is then incorporated into containers, such as tubes, which are suitable for topical administration.

### Example 6G

To prepare a pharmaceutical topical gel composition, about 100 μg of formoterol and about 100 mg of budesonide is mixed with 1.75 g of hydroxypropyl cellulose, 10 ml of propylene glycol, 10 ml of isopropyl myristate, and 100 ml of purified alcohol USP. The resulting gel mixture is then incorporated into containers, such as tubes, which are suitable for topical administration.

### Example 6H

To prepare a pharmaceutical topical gel composition, about 100 mg of salmeterol is mixed with about 10 ml of PEG-400, 1.75 g of hydroxypropyl cellulose, 10 ml of isopropyl myristate and 100 ml of purified alcohol USP. The resulting gel mixture is then incorporated into containers, such as tubes, which are suitable for topical administration.

### Example 6I

To prepare a pharmaceutical topical gel composition, about 100 mg of prednisolone is mixed with about 10 ml of PEG-400, 1.75 g of hydroxypropyl cellulose, 10 ml of isopropyl myristate, and 100 ml of purified alcohol USP. The resulting gel mixture is then incorporated into containers, such as tubes, which are suitable for topical administration.

Oral Compositions

### Example 6J

To prepare a pharmaceutical oral composition, about 100 mg of a compound of prednisolone is mixed with 750 mg of starch. The mixture is incorporated into an oral dosage unit for, such as a hard gelatin capsule, which is suitable for oral administration.

### Example 6K

To prepare a pharmaceutical oral composition, about 50 mg of a compound of budesonide is mixed with 375 mg of gelatin. The mixture is incorporated into an oral dosage unit for, such as a hard gelatin capsule, which is suitable for oral administration.

### Example 6L

To prepare a pharmaceutical oral composition, about 200 mg of a compound of ketotifen and 100 μg of formoterol is mixed with 1500 mg of hydroxypropylmethylcellulose. The mixture is incorporated into an oral dosage unit for, such as a hard gelatin capsule, which is suitable for oral administration.

### Example 6M

To prepare a pharmaceutical oral composition, about 50 mg of a compound of fluticasone propionate is mixed with 600 mg of starch. The mixture is incorporated into an oral dosage unit for, such as a hard gelatin capsule, which is suitable for oral administration.

### Example 7

### Beta Adrenergic Agonists and Glucocorticosteroid Administration Regimens

Non-limiting examples of such administration regimens are as follows:

### Example 7A

A patient having a regional fat deposit or lipoma is administered a therapeutically effective amount of composition 6A on day 1 and every subsequently odd-numbered day of treatment. For a lipoma 0.2 to 0.5 ml of formulation is injected into the center of the lipoma. More than one injection may be performed for lipomas of size greater than 2 cm. For regional fat accumulations 5 to 20 injections of 0.1 to 0.2 ml of formulation 6A is administered over a region (e.g. lateral abdomen). Each injection of 0.1 to 0.5 ml will treat a 1 to 5 cm² of fat.

### Example 7B

A patient having a regional fat deposit or lipoma is administered a therapeutically effective amount of composition 6C and 6D on day 1 and every 3 to 7 days thereafter.

### Example 7C

A patient having a regional fat deposit or lipoma is administered a therapeutically effective amount of composition 6D on day 1 and composition 6C and 6D are co administered every 3 to 7 days thereafter.

### Example 7D

A patient having a regional fat deposit or lipoma is administered a therapeutically effective amount of composition 6E every 3 to 7 days.

### Example 7E

A patient having a regional fat deposit or lipoma is administered a therapeutically effective amount of composition 6D on day 1 followed by a holiday of two days. On the second holiday, the patient is administered a therapeutically effective amount of composition 6C. This administration is then repeated.

### Example 7F

A patient having a regional fat deposit or lipoma is administered a therapeutically effective amount of composition 6D on day 1 followed by a holiday of two days. On the second holiday, the patient is administered a therapeutically effective amount of composition 6C. This administration is then repeated.

### Example 7G

A patient having a regional fat deposit or lipoma is administered a therapeutically effective amount of composition 61 on day 1 and every subsequently odd-numbered day of treatment. On even-numbered days, the patient is administered a therapeutically effective amount of composition 6C.

### Example 7H

A patient having a regional fat deposit or lipoma is administered a therapeutically effective amount of composition 6C daily. On even-numbered days, the patient is administered a therapeutically effective amount of composition 6I.

### Example 7I

A patient with obstructive sleep apnea is administered a therapeutically effective amount of composition 6A bilaterally into the soft palate and posterior and lateral pharynx on day on and every other day thereafter. Approximately 0.1 to 0.5 cc are administered. Each injection of 0.1 to 0.5 cc will treat approximately 1 to 5 cm² of fat.

### Example 7J

A patient with obstructive sleep apnea is administered a therapeutically effective amount of composition 6E bilaterally into the soft palate and posterior and lateral pharynx on day 1 and every 3 to 7 days thereafter until alleviation of symptoms. Approximately 0.1 to 0.5 cc are administered. Each injection of 0.1 to 0.5 cc will treat an approximate 1 to 5 cm² of fat.

### Example 7K

A patient with a muscle injury such as a tear is administered a therapeutically effective amount of composition 6E into the injured area on day one and every 3 to 7 days thereafter.

### Example 7L

A patient with muscle wasting or cachexia is administered a therapeutically effective amount of 6L is administered to the patient twice daily.

### Example 7M

A patient with psoriasis applies a therapeutically effective amount of 6F to the skin area having a psoriatic plaque.

The examples and embodiments described herein are for illustrative purposes only and various modifications or changes are included within the spirit and purview of this application and scope of the appended claims. All publications, patents, and patent applications cited herein are hereby incorporated by reference for all purposes.

1. A method for reducing a regional fat deposit in a subject in need thereof comprising administering to the subject, a sustained release pharmaceutical composition comprising a therapeutically effective amount of at least one compound for reducing desensitization of beta adrenergic receptors wherein the regional fat deposit in the subject is reduced.

2. The method of claim 1, wherein the sustained release pharmaceutical composition is administered by a parenteral, topical, intramuscular, subcutaneous, or transdermal route of administration.

3. The method of claim 1, wherein the at least one compound comprises a glucocorticosteroid, an antihistamine, or a combination thereof.

4. The method of claim 3, wherein the at least one compound comprises dexamethasone, prednisolone, methylprednisolone, fluticasone propionate, budesonide, ketotifen, or any combination thereof.

5. The method of claim 1, wherein the therapeutically effective amount of the at least one compound comprises a crystalline microparticle suspension of the at least one compound.

6. (canceled)

7. (canceled)

8. The method of claim 1, wherein the sustained release pharmaceutical composition further comprises a therapeutically effective amount of at least one beta adrenergic agonist that is selective for the beta-2 adrenergic receptor, and wherein the beta adrenergic agonist is formulated as a crystalline microparticle suspension.

9. (canceled)

10. The method of claim 8, wherein the therapeutically effective amount of the at least one beta adrenergic agonist is in solubilized form.

11. (canceled)

12. (canceled)

13. (canceled)

14. (canceled)

15. (canceled)

16. The method of claim 10, wherein the at least one beta adrenergic agonist comprises salmeterol, formoterol, bambuterol, eformoterol, isoproterenol, albuterol, fenoterol, or any combination thereof.

17. (canceled)

18. (canceled)

19. (canceled)

20. (canceled)

21. A method for reducing a regional fat deposit in a subject in need thereof comprising administering to the subject:
(a) a therapeutically effective amount of one or more adrenergic receptor pathway-stimulating compounds; and
(b) a therapeutically effective amount of at least one compound for reducing beta adrenergic receptor desensitization.

22. The method of claim 21, wherein the one or more adrenergic receptor pathway-stimulating compounds comprise a catecholamine, an alpha adrenergic antagonist, forskolin, aminophylline, analogs thereof, or any combination thereof.

23. The method of claim 21, further comprising administering a composition comprising a therapeutically effective amount of at least one beta adrenergic agonist that is selective for the beta-2 adrenergic receptor.

24. (canceled)

25. (canceled)

26. (canceled)

27. (canceled)

28. A sustained release pharmaceutical composition comprising a therapeutically effective amount of at least one compound for reducing desensitization of beta adrenergic receptors, wherein the therapeutically effective amount of the at least one compound comprises a crystalline microparticle suspension of the at least one compound and wherein the composition provides a sustained release form.

29. The sustained release pharmaceutical composition of claim 28, wherein the at least one compound is a glucocorticosteroid, an antihistamine, or any combination thereof.

30. The sustained release pharmaceutical composition of claim 29, wherein the at least one compound comprises dexamethasone, prednisolone, methylprednisolone, fluticasone propionate, budesonide, ketotifen, or any combination thereof.

31. The sustained release pharmaceutical composition of claim 28, further comprising a crystalline microparticle suspension of at least one beta adrenergic agonist that is selective for the beta-2 adrenergic receptor.

32. The sustained release pharmaceutical composition of claim 31, wherein the therapeutically effective amount of the at least one beta adrenergic agonist in solubilized form.

33. The sustained release pharmaceutical composition of claim 31, wherein the at least one beta adrenergic agonist comprises salmeterol, formoterol, bambuterol, eformoterol, isoproterenol, albuterol, or fenoterol, or any combination thereof.

34. (canceled)

35. The sustained release pharmaceutical composition of claim 28, wherein the sustained release pharmaceutical composition is formulated for injection.

36. (canceled)

37. (canceled)

38. (canceled)

39. (canceled)

40. (canceled)

41. The method of claim 16 wherein the sustained release pharmaceutical composition is administered at or near the regional fat deposit wherein the regional fat deposit is found in lipomas, the palate area, the pharynx area, and the region of the neck.

42. The method of claim 41 wherein the method treats psoriasis or hypopigmentation.
